(19) United States
(12) Patent Application Publication
(10) Pub. No.: US 2012/0107261 A1
(43) Pub. Date: May 3, 2012

(54) COSMETIC COMPOSITIONS HAVING SUSPENSIONS OF SILVER SALTS

(76) Inventors: Bernhard Banowski, Dusseldorf (DE); Imme Breuer, Dusseldorf (DE); Nadine Buse, Hilden (DE); Barbara Heide, Krefeld (DE); Gertraud Teckenbrock, Sprockhovel (DE); Brend Añderheggen, Monchengladbach (DE)

(21) Appl. No.: 13/345,828

(22) Filed: Jan. 9, 2012

Related U.S. Application Data
(63) Continuation of application No. PCT/EP2010/057209, filed on May 26, 2010.

(30) Foreign Application Priority Data
Jul. 10, 2009 (DE) 10 2009 027 604.1

(51) Int. Cl. A61K 8/25; A61K 8/46; A61K 8/27; A61Q 15/00; A61K 8/26; A61K 8/29; A61K 8/34

(52) U.S. Cl. 424/67; 424/65; 424/68

(57) ABSTRACT

An anhydrous cosmetic or dermatological preparation includes an aqueous suspension of at least one silver halide having a water content of up to 90 wt. %, relative to the total mass of the suspension, applied to a carrier, in combination with one or more passivating agents selected from the group of phyllosilicates and/or talc, one or more antiperspirant active ingredients, and perfume.

### CROSS-REFERENCES TO RELATED APPLICATIONS

This application is a continuation of PCT/EP2010/057209, filed on May 26, 2010, which claims priority under 35 U.S.C. §119 to DE 10 2009 027 604.1 filed on Jul. 10, 2009, both of which are hereby incorporated by reference.

### FIELD OF THE INVENTION

The present invention generally relates to anhydrous cosmetic or dermatological preparations comprising aqueous suspensions of at least one silver halide applied to a carrier, in combination with passivating agents selected from the group of phyllosilicates or talc.

### BACKGROUND OF THE INVENTION

The use of silver as a disinfectant in wound treatment, medical devices and in pharmaceutical and cosmetic preparations is known in the prior art. For cosmetic agents, for example, a maximum content of 4% silver nitrate is permitted (German Cosmetics Ordinance, Annex 3 to §3, Part A).

In contrast to aqueous solutions of soluble silver salts (e.g. silver nitrate or silver acetate), aqueous colloidal silver solutions are either liquid dispersions of elemental silver or liquid dispersions of complex poorly soluble silver compounds. The water-dispersible starting materials then contain only traces of silver ions.

There is a need for non-colloidal silver for cosmetic preparations.

WO 2006029213 A1 describes silver citrate compounds having an antimicrobial action. These silver compounds stabilized in aqueous citric acid are available commercially under the name TINOSAN® SDC. The production of these compounds by means of an electrochemical process is described in EP 1 041 879 A1. TINOSAN® SDC encompasses silver ions (Ag⁺) produced by electrolysis, and the mixture contains no colloidal silver.

The TINOSAN® SDC described in the publications WO 2006029213 A1 and EP 1041879 A1 can be used in cosmetic, aqueous and/or emulsion-based compositions and with various additives. In particular, deodorants and antiperspirants in combination with TINOSAN® SDC are described in WO 2006029213 A1.

The use of silver compounds dissolved in water has proved problematic, however, because of corrosion in metallic storage means such as aerosol cans. This effect is heightened by the presence of corrosion-promoting compounds, such as for example acid aluminum salts and/or aluminum zirconium salts. In addition, contact with water often leads to clogging of the valves in the aerosol and to agglomeration, instabilities and inhomogeneities in other cosmetic application forms.

A further problem with hydrous mixtures can occur with the use of water-soluble encapsulated perfume. In the presence of water these encapsulated perfumes can swell, stick together, and react by disintegrating.

In order to solve these problems, DE 202008014407 U1 proposes the use of one or more passivating agents selected from the group of phyllosilicates and/or talc. Agents containing up to 10 wt. % of water (a maximum of 5 wt. % from the TINOSAN® SDC plus a further 5 wt. % from other sources) can be provided in this way.

Starting gelation of a phyllosilicate by contact with an aqueous silver salt solution in the presence of an electrolyte such as citric acid to produce a gel having an antibacterial action was likewise already known from US 20050266081 A1 .

There was also a demand for ways of stably incorporating silver into cosmetic compositions, even with water contents of well below 10 wt. %. Even in systems having substantially lower water content, where TINOSAN® SDC cannot be used, the application of antimicrobial silver compounds dissolved in water from an intrinsically hydrophobic environment should be made possible.

### BRIEF SUMMARY OF THE INVENTION

Solutions to the above-mentioned problems, and others, are met by an anhydrous cosmetic or dermatological preparation, comprising an aqueous suspension of at least one silver halide having a water content of up to 90 wt. %, relative to the total mass of the suspension, applied to a carrier, in combination with one or more passivating agents selected from the group of phyllosilicates and/or talc, one or more antiperspirant active ingredients, and perfume.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

According to the invention, “anhydrous preparation” means that in addition to the water contained in the silver suspension, amounting to up to 90 wt. % relative to the total mass of the aqueous suspension of at least one silver halide applied to a carrier, additional water can be included only up to a proportion of approximately 3 wt. % relative to the total mass of the preparation. Anhydrous preferably means a content of a maximum of 2 wt. %, most preferably about 0 wt. %, relative to the total mass of the preparation. In addition to the water introduced by the aqueous suspension of at least one silver halide applied to a carrier, the preparation therefore contains a maximum of 3 wt. % of further water, relative to the total mass of the preparation.

With a preferred maximum proportion of silver suspension of approximately 5 wt. % and a maximum water content within said suspension of 90 wt. %, the preparation according to the invention accordingly preferably encompasses a maximum of 4 to 5 wt. % of water that can be introduced by the suspension. In addition, a maximum of 3 wt. % of further water is accepted in the preparation without giving rise to problems such as are known in the prior art. According to the invention the preparation can therefore contain in total a maximum water content of 8 wt. %, formed from water from the suspension and water from other constituents of the preparation or deliberately added water, without this resulting in a diminishing of the advantages of the preparation according to the invention as described below. Despite this possible proportion of water, the preparation according to the invention is understood to be anhydrous. The advantages are set out below.

The invention makes it possible for this aqueous silver suspension with its antimicrobial action to be used in an environment that in the presence of water normally leads to instabilities and storage problems, as previously stated. For example, the use of the aqueous silver suspension in aerosol preparations would be critical, since water can corrode the metal can or valve components, e.g. the spring. This effect is heightened by the presence of corrosion-promoting compounds, such as for example acid aluminum salts and/or aluminum zirconium salts, which are conventionally used in antiperspirant or deodorant preparations.

The aqueous suspension of at least one silver halide applied to a carrier (also referred to as “silver suspension”), contained according to the invention in the agents, contains at least one silver halide, with silver chloride, silver bromide and silver iodide being preferred. Mixtures of silver halides can of course also be applied to one or more carrier materials. Silver chloride is most particularly preferred, such that preferred preparations encompass an aqueous suspension of silver chloride applied to a carrier.

Silver is preferably used in these suspensions in defined amounts that are guided by the amount of suspension used and the resulting total amount of silver in the cosmetic composition. Preferred compositions according to the invention have the characterizing feature that the aqueous suspension of at least one silver halide applied to a carrier contains, relative to the weight of the suspension, 0.1 to 10 wt. %, preferably 0.5 to 7.5 wt. %, more preferably 1 to 5 wt. % and in particular 1.5 to 2.5 wt. %, of silver (calculated as Ag⁺).

As has already been mentioned, silver chloride is particularly preferably used, such that particularly preferred compositions according to the invention have the characterizing feature that the aqueous suspension of at least one silver halide applied to a carrier contains, relative to the weight of the suspension, 0.1 to 10 wt. %, preferably 0.5 to 7.5 wt. %, more preferably 1 to 5 wt. % and in particular 1.5 to 2.5 wt. %, of silver chloride.

Silver has a molar mass of 107.87 g/mol. Silver chloride has a molar mass of 143.32 g/mol. Preferred compositions according to the invention have the characterizing feature that silver chloride is included in a total amount from 1.3 to 133 ppm, preferably 2.6 to 66.4 ppm, particularly preferably 6.6 to 26.6 ppm, exceptionally preferably 9.3 to 13.3 ppm, relative in each case to the weight of the propellant-free composition according to the invention.

Silver bromide has a molar mass of 187.77 g/mol. Further preferred compositions according to the invention have the characterizing feature that silver bromide is included in a total amount from 1.7 to 174 ppm, preferably 3.4 to 87 ppm, particularly preferably 8.7 to 35 ppm, exceptionally preferably 12.2 to 17.4 ppm, relative in each case to the weight of the propellant-free composition according to the invention.

Silver iodide has a molar mass of 234.773 g/mol. Further preferred compositions according to the invention have the characterizing feature that silver iodide is included in a total amount from 2.2 to 218 ppm, preferably 4.4 to 109 ppm, particularly preferably 11 to 43.5 ppm, exceptionally preferably 15.2 to 22 ppm, relative in each case to the weight of the propellant-free composition according to the invention.

Further preferred compositions according to the invention have the characterizing feature that silver ions (Ag⁺) are included in a total amount from 1 to 100 ppm, preferably 2 to 50 ppm, particularly preferably 5 to 20 ppm, exceptionally preferably 7 to 10 ppm, relative in each case to the weight of the propellant-free composition according to the invention.

The mixture of active ingredients is the same as the preparation, but if the preparation is an aerosol, the proportions relate to the active mixture excluding propellant.

Water-insoluble, inert materials such as metal oxides, zeolites, etc., are preferably used as carrier materials for the silver halide(s). In the context of the present invention titanium dioxide in particular has proved to be an outstanding carrier material, such that preferred preparations encompass an aqueous suspension of at least one silver halide applied to titanium dioxide. Particularly preferred preparations according to the invention encompass an aqueous suspension of a silver chloride applied to titanium dioxide.

The suspensions in turn preferably contain defined amounts of carrier material. Compositions according to the invention are preferred here in which the aqueous suspension of at least one silver halide applied to a carrier contains, relative to the weight of the suspension, 0.1 to 20 wt. %, preferably 1 to 17.5 wt. %, more preferably 2.5 to 15 wt. % and in particular 5 to 10 wt. %, of titanium dioxide.

It has been found that the suspension can be effectively stabilized by means of sulfosuccinates in particular. Moreover, an appropriate content of sulfosuccinates makes it easier to incorporate the suspension into the agents according to the invention. In addition, these high-foaming, gentle surfactants have a positive influence in the application of the cosmetic. Particularly preferred compositions according to the invention therefore encompass an aqueous suspension of at least one silver halide applied to a carrier, which suspension additionally contains, relative to the weight of the suspension, 0.1 to 30 wt. %, preferably 0.5 to 27.5 wt. %, more preferably 1 to 25 wt. % and in particular 5 to 20 wt. %, of at least one sulfosuccinate.

Among the compounds in this group, certain representatives have proved to be particularly suitable. Most particularly preferred compositions according to the invention have the characterizing feature that the aqueous suspension of at least one silver halide applied to a carrier contains, relative to the weight of the suspension, 0.1 to 30 wt. %, preferably 0.5 to 27.5 wt. %, more preferably 1 to 25 wt. % and in particular 5 to 20 wt. %, of sodium di(2-ethylhexyl)sulfosuccinate

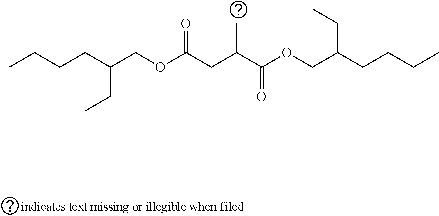

It has further been found that a content of diols in the suspension improves the stability and incorporability of the suspension, with short-chain diols preferably being used. Most particularly preferred compositions according to the invention have the characterizing feature that the aqueous suspension of at least one silver halide applied to a carrier contains, relative to the weight of the suspension, 0.1 to 10 wt. %, preferably 0.5 to 7.5 wt. %, more preferably 1 to 5 wt. % and in particular 2.5 to 5 wt. %, of propylene glycol.

Water can be contained in the silver suspension used according to the invention in amounts of up to 90 wt. % (relative to the suspension). Suspensions having a lower water content are preferably used; most particularly preferred compositions have the characterizing feature that the aqueous suspension of at least one silver halide applied to a carrier contains, relative to the weight of the suspension, 30 to 90 wt. %, preferably 40 to 85 wt. %, more preferably 50 to 80 wt. % and in particular 60 to 70 wt. %, of water.

According to the invention, the water that is present because of the silver suspension together with any additional water (max. 3 wt. %) is complexed and bonded by the passivating agent. The water supplied via the suspension is bonded to the passivating agent and is therefore no longer available for a damaging influence. The passivating agents are preferably selected from the group of phyllosilicates and from talc. Phyllosilicates are polymeric crystalline sodium disilicates. Phyllosilicates that are preferred according to the invention are selected from clay minerals such as bentonite, montmorillonite, nontronite, hectorite, saponite, sauconite, beidellite, allevardite, illite, halloysite, attapulgite, muscovite, serpentine, kaolinite, pyrophyllite, smectite, glauconite, vermiculite and/or sepiolite.

In a particularly preferred embodiment the at least one passivating agent is hydrophobically modified. A correspondingly preferred hydrophobically modified phyllosilicate passivating agent is disteardimonium hectorite. Hectorites are a monoclinic clay mineral similar to montmorillonite and belonging to the smectites, with the formula M_{0.3}⁺(Mg_{2.7}Li_{0.3}) [Si₄O₁₀(OH)₂], where M⁺ is usually Na⁺. Another passivating agent according to the invention is talc, preferably talc with the INCI name Talc, E553b. Talc is a natural, widely occurring magnesium silicate Mg₃[(OH)₂/Si₄O₁₀] or 3.MgO.4 SiO₂.H₂O belonging to the 2:1-type phyllosilicates, denser aggregates of which are called soapstone.

According to the invention it has now been found that, contrary to the expected findings, these passivating agents bond the water in the aqueous suspension of at least one silver halide applied to a carrier only to the extent that no negative side-effects such as corrosion, etc., occur.

In the case of silver passivated with phyllosilicates, this effect is achieved in particular in the presence of sweat, since the sodium ions contained therein help to displace the silver from the bonding sites in the phyllosilicate. It has been found that the silver is incorporated into the phyllosilicates or the talc. However, the antimicrobial activity is not lost in this way but is rather reactivated via the release of the silver in contact with water. This mechanism of action according to the invention is advantageous for deodorants or antiperspirants for example, in that the antimicrobial action occurs only as a result of perspiration at the active site.

The combination according to the invention is advantageous for aerosol preparations in particular, as the water from the aqueous suspension of at least one silver halide applied to a carrier is used to active phyllosilicates and is bonded to them. As a result of this specific process, the unpleasant side-effects do not occur. The antimicrobial activity, such as for example the deodorizing capacity, is thus retained even though the silver halide deodorizing active ingredient or the water suspending it combines closely with the phyllosilicates.

In the prior art phyllosilicates are normally activated prior to their use, i.e. they are converted to a swellable form by treating the phyllosilicates with a polar liquid and high shear forces. For anhydrous antiperspirant aerosols, propylene carbonate, ethanol or dipropylene glycol for example are used as polar liquids. This activation can be dispensed with according to the invention. According to the invention activation is now performed with the involvement of the aqueous suspension of at least one silver halide applied to a carrier. The phyllosilicate activation can advantageously be optimized by adding additional perfume and the solvents contained therein. The phyllosilicates are activated in this way, which in turn leads to a rise in the viscosity of the complete preparation and the phyllosilicates precipitate out less easily and more slowly. The water content is completely bonded to the phyllo silicate and cannot give rise to negative effects (corrosion, agglomeration of perfume capsules, etc.). Furthermore, the silver itself surprisingly is incorporated into the phyllosilicates and is only actively released on contact with the sweat on the skin.

By contrast, a conventional production process, in which the silver deodorizing active ingredient, an aqueous suspension of at least one silver halide applied to a carrier, is added only after activation of the phyllosilicates by intensive shearing, leads to the known disadvantages, as in this case the bonding sites of the phyllosilicates are occupied by other constituents of the formula and are no longer available in sufficient numbers for the water molecules of the aqueous suspension of at least one silver halide applied to a carrier.

An identical effect was established according to the invention with anhydrous antiperspirant sticks too. In the case of anhydrous antiperspirant sticks according to the invention the water is bonded not to silicates but to talc, and surprisingly the same advantages are observed. The active release of the silver occurs only on contact with water, in other words it is only triggered by the sweat. The bonding of the water to the fillers, such as talc, in the antiperspirant stick prevents the water-soluble antiperspirant salt, preferably aluminum chlorohydrate (ACH) or aluminum zirconium salts (AZG), that is likewise present in powder form from being dissolved and/or from agglomerating.

The application of stick material containing such agglomerates can lead to a negatively perceived sensory perception and to increased residues on the skin and clothing. This is avoided according to the invention.

The combination of an aqueous suspension of at least one silver halide applied to a carrier and a passivating agent makes it possible to produce cosmetic or dermatological preparations that develop the desired antimicrobial activity at the active site only through external addition of water.

Cosmetic antiperspirants or deodorants serve to eliminate body odor that arises when inherently odorless, fresh sweat is broken down by microorganisms. Conventional cosmetic deodorants are based on a number of different modes of action. In antiperspirants the formation of sweat can be reduced by astringents, predominantly aluminum salts, such as aluminum hydroxychloride (aluminum chlorohydrate (ACH), which can be activated (AACH)) or aluminum zirconium salts (AZG). The bacteria flora on the skin can be reduced by the use of antimicrobial silver salt applied to a carrier in cosmetic antiperspirants. The microorganisms giving rise to the odor are effectively reduced in this way. The flow of sweat itself is not influenced; ideally only the microbial decomposition of the sweat is temporarily halted.

The compositions according to the invention contain at least one antiperspirant active ingredient.

Preferred antiperspirant active ingredients are selected from the water-soluble astringent inorganic and organic salts of aluminum, zirconium and zinc and any mixtures of these salts.

According to the invention water solubility is understood to mean a solubility of at least 5 wt. % at 20° C., in other words amounts of at least 5 g of the antiperspirant active ingredient are soluble in 95 g of water at 20° C.

Particularly preferred antiperspirant active ingredients are selected from aluminum chlorohydrate, in particular aluminum chlorohydrate of the general formula [Al₂(OH)₅Cl.1-6H₂O]ₙ, preferably [Al₂(OH)₅Cl.2-3H₂O]ₙ, which can be present in non-activated or in activated (depolymerized) form, and aluminum chlorohydrate of the general formula [Al₂(OH)₄Cl₂.1-6H₂O]ₙ, preferably [Al₂(OH)₄Cl₂.2-3H₂O]ₙ, which can be present in non-activated or in activated (depolymerized) form.

The production of preferred antiperspirant active ingredients is disclosed for example in U.S. Pat. No. 3,887,692, U.S. Pat. No. 3,904,741, U.S. Pat. No. 4,359,456, GB 2048229 and GB 1347950.

Also preferred are aluminum sesquichlorohydrate, aluminum dichlorohydrate, aluminum chlorohydrex propylene glycol (PG) or aluminum chlorohydrex polyethylene glycol (PEG), aluminum or aluminum zirconium glycol complexes, e.g. aluminum or aluminum zirconium propylene glycol complexes, aluminum sesquichlorohydrex PG or aluminum sesquichlorohydrex PEG, aluminum PG dichlorohydrex or aluminum PEG dichlorohydrex, aluminum hydroxide, selected furthermore from aluminum zirconium chlorohydrates, such as aluminum zirconium trichlorohydrate, aluminum zirconium tetrachlorohydrate, aluminum zirconium pentachlorohydrate, aluminum zirconium octachlorohydrate, aluminum zirconium chlorohydrate glycine complexes such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAl(SO₄)₂.12H₂O, alum), aluminum undecylenoyl collagen amino acid, sodium aluminum lactate+aluminum sulfate, sodium aluminum chlorohydroxylactate, aluminum bromohydrate, aluminum chloride, complexes of zinc and sodium salts, complexes of lanthanum and cerium, aluminum salts of lipoamino acids, aluminum sulfate, aluminum lactate, aluminum chlorohydroxyallantoinate, sodium aluminum chlorohydroxylactate, zinc chloride, zinc sulfocarbolate, zinc sulfate, zirconyl oxyhalides, in particular zirconyl oxychlorides, zirconyl hydroxyhalides, in particular zirconyl hydroxychlorides (zirconium chlorohydrate).

Antiperspirant active ingredients that are particularly preferred according to the invention are selected from “activated” aluminum and aluminum zirconium salts, which are also referred to as antiperspirant active ingredients with enhanced activity. Such active ingredients are known in the prior art and are also available commercially. Their production is disclosed in GB 2048229, U.S. Pat. No. 4,775,528 and US 6010688, for example. Activated aluminum and aluminum zirconium salts are generally produced by heat treating a relatively dilute solution of the salt (e.g. approximately 10 wt. % salt) to increase its HPLC peak 4 to peak 3 surface area ratio. The activated salt can then be dried, in particular spray-dried, to form a powder. In addition to spray drying, drum drying for example is also suitable.

Activated aluminum and aluminum zirconium salts typically have an HPLC peak 4 to peak 3 surface area ratio of at least 0.4, preferably at least 0.7, particularly preferably at least 0.9, wherein at least 70% of the aluminum can be assigned to these peaks. Activated aluminum and aluminum zirconium salts do not necessarily have to be used as a spray-dried powder. Sweat-inhibiting active ingredients that are likewise preferred according to the invention are non-aqueous solutions or solubilizates of an activated sweat-inhibiting aluminum or aluminum zirconium salt, for example according to US 6010688, which are stabilized against loss of activation by the rapid degradation of the HPLC peak 4 to peak 3 surface area ratio of the salt by the addition of an effective amount of a polyhydric alcohol having 3 to 6 carbon atoms and 3 to 6 hydroxyl groups, preferably propylene glycol, sorbitol and pentaerythritol. Compositions are preferred for example that contain in percent by weight (USP): 18 to 45 wt. % of an activated aluminum or aluminum zirconium salt, 55 to 82 wt. % of at least one anhydrous polyhydric alcohol having 3 to 6 carbon atoms and 3 to 6 hydroxyl groups, preferably propylene glycol, butylene glycol, diethylene glycol, dipropylene glycol, glycerol, sorbitol and pentaerythritol, particularly preferably propylene glycol.

Complexes of activated sweat-inhibiting aluminum or aluminum zirconium salts with a polyhydric alcohol containing 20 to 50 wt. %, particularly preferably 20 to 42 wt. %, of activated sweat-inhibiting aluminum or aluminum zirconium salt and 2 to 16 wt. % of molecularly bonded water are also particularly preferred, wherein the remainder up to 100 wt. % is made up by at least one polyhydric alcohol having 3 to 6 carbon atoms and 3 to 6 hydroxyl groups. Propylene glycol, propylene glycol/sorbitol mixtures and propylene glycol/pentaerythritol mixtures are preferred alcohols of this type. Such complexes of an activated sweat-inhibiting aluminum or aluminum zirconium salt with a polyhydric alcohol that are preferred according to the invention are disclosed in U.S. Pat. No. 5,643,558 and U.S. Pat. No. 6,245,325 for example.

Further preferred sweat-inhibiting active ingredients are basic calcium aluminum salts such as are disclosed in US 2571030 for example. These salts are produced by reacting calcium carbonate with aluminum chlorohydroxide or aluminum chloride and aluminum powder or by adding calcium chloride dihydrate to aluminum chlorohydroxide.

Further preferred sweat-inhibiting active ingredients are aluminum zirconium complexes such as are disclosed in U.S. Pat. No. 4,017,599 for example, which are buffered with salts of amino acids, in particular with alkali and alkaline-earth glycinates.

Further preferred sweat-inhibiting active ingredients are activated aluminum or aluminum zirconium salts, such as are disclosed in U.S. Pat. No. 6,245,325 or U.S. Pat. No. 6,042,816 for example, containing 5 to 78 wt. % (USP) of an activated sweat-inhibiting aluminum or aluminum zirconium salt, an amino acid or hydroxyalkanoic acid in an amount to provide a weight ratio of (amino acid or hydroxyalkanoic acid) to (Al+Zr) of 2:1 to 1:20 and preferably 1:1 to 1:10, and a water-soluble calcium salt in an amount to provide a weight ratio of Ca to (Al+Zr) of 1:1 to 1:28 and preferably 1:2 to 1:25.

Particularly preferred solid activated sweat-inhibiting salt compositions, according to U.S. Pat. No. 6,245,325 or U.S. Pat. No. 6,042,816 for example, contain 48 to 78 wt. % (USP), preferably 66 to 75 wt. % of an activated aluminum or aluminum zirconium salt and 1 to 16 wt. %, preferably 4 to 13 wt. % of molecularly bonded water (water of hydration), also sufficient water-soluble calcium salt that the weight ratio of Ca to (Al+Zr) is 1:1 to 1:28, preferably 1:2 to 1:25, and sufficient amino acid that the weight ratio of amino acid to (Al+Zr) is 2:1 to 1:20, preferably 1:1 to 1:10.

Further particularly preferred solid sweat-inhibiting activated salt compositions, according to U.S. Pat. No. 6,245,325 or U.S. Pat. No. 6,042,816 for example, contain 48 to 78 wt. % (USP), preferably 66 to 75 wt. % of an activated aluminum or aluminum zirconium salt and 1 to 16 wt. %, preferably 4 to 13 wt. % of molecularly bonded water (water of hydration), also sufficient water-soluble calcium salt that the weight ratio of Ca to (Al+Zr) is 1:1 to 1:28, preferably 1:2 to 1:25, and sufficient glycine that the weight ratio of glycine to (Al+Zr) is 2:1 to 1:20, preferably 1:1 to 1:10.

Further particularly preferred solid sweat-inhibiting activated salt compositions, according to U.S. Pat. No. 6,245,325 or U.S. Pat. No. 6,042,816 for example, contain 48 to 78 wt. % (USP), preferably 66 to 75 wt. % of an activated aluminum or aluminum zirconium salt and 1 to 16 wt. %, preferably 4 to 13 wt. % of molecularly bonded water, also sufficient water-soluble calcium salt that the weight ratio of Ca to (Al+Zr) is 1:1 to 1:28, preferably 1:2 to 1:25, and sufficient hydroxyalkanoic acid that the weight ratio of hydroxyalkanoic acid to (Al+Zr) is 2:1 to 1:20, preferably 1:1 to 1:10.

Preferred water-soluble calcium salts for stabilizing the sweat-inhibiting salts are selected from calcium chloride, calcium bromide, calcium nitrate, calcium citrate, calcium formate, calcium acetate, calcium gluconate, calcium ascorbate, calcium lactate, calcium glycinate, calcium carbonate, calcium sulfate, calcium hydroxide, and mixtures thereof.

Preferred amino acids for stabilizing the sweat-inhibiting salts are selected from glycine, alanine, leucine, isoleucine, β-alanine, valine, cysteine, serine, tryptophane, phenylalanine, methionine, β-amino-n-butanoic acid and γ-amino-n-butanoic acid and the salts thereof, in each case in the d form, 1 form or dl form; glycine is particularly preferred.

Preferred hydroxyalkanoic acids for stabilizing the sweat-inhibiting salts are selected from glycolic acid and lactic acid.

Further preferred sweat-inhibiting active ingredients are activated aluminum or aluminum zirconium salts, such as are disclosed in U.S. Pat. No. 6,902,723 for example, containing 5 to 78 wt. % (USP) of an activated sweat-inhibiting aluminum or aluminum zirconium salt, an amino acid or hydroxyalkanoic acid in an amount to provide a weight ratio of (amino acid or hydroxyalkanoic acid) to (Al+Zr) of 2:1 to 1:20 and preferably 1:1 to 1:10, and a water-soluble strontium salt in an amount to provide a weight ratio of Sr to (Al+Zr) of 1:1 to 1:28 and preferably 1:2 to 1:25.

Particularly preferred solid sweat-inhibiting activated salt compositions, according to U.S. Pat. No. 6,902,723 for example, contain 48 to 78 wt. % (USP), preferably 66 to 75 wt. % of an activated aluminum or aluminum zirconium salt and 1 to 16 wt. %, preferably 4 to 13 wt. % of molecularly bonded water, also sufficient water-soluble strontium salt that the weight ratio of Sr to (Al+Zr) is 1:1 to 1:28, preferably 1:2 to 1:25, and sufficient amino acid that the weight ratio of amino acid to (Al+Zr) is 2:1 to 1:20, preferably 1:1 to 1:10.

Further particularly preferred solid sweat-inhibiting activated salt compositions, according to U.S. Pat. No. 6,902,723 for example, contain 48 to 78 wt. % (USP), preferably 66 to 75 wt. % of an activated aluminum or aluminum zirconium salt and 1 to 16 wt. %, preferably 4 to 13 wt. % of molecularly bonded water, also sufficient water-soluble strontium salt that the weight ratio of Sr to (Al+Zr) is 1:1 to 1:28, preferably 1:2 to 1:25, and sufficient glycine that the weight ratio of glycine to (Al+Zr) is 2:1 to 1:20, preferably 1:1 to 1:10.

Further particularly preferred solid sweat-inhibiting activated salt compositions, according to U.S. Pat. No. 6,902,723 for example, contain 48 to 78 wt. % (USP), preferably 66 to 75 wt. % of an activated aluminum or aluminum zirconium salt and 1 to 16 wt. %, preferably 4 to 13 wt. % of molecularly bonded water, also sufficient water-soluble strontium salt that the weight ratio of Sr to (Al+Zr) is 1:1 to 1:28, preferably 1:2 to 1:25, and sufficient hydroxyalkanoic acid that the weight ratio of hydroxyalkanoic acid to (Al+Zr) is 2:1 to 1:20, preferably 1:1 to 1:10.

Further preferred activated aluminum salts are those of the general formula Al₂(OH)₆₋ₐXa, in which X is Cl, Br, l or NO₃ and “a” is a value from 0.3 to 5, preferably from 0.8 to 2.5 and particularly preferably 1 to 2, such that the molar ratio of Al to X is 0.9:1 to 2.1:1, as is disclosed in U.S. Pat. No. 6,074,632 for example. These salts generally contain a little associatively bonded water of hydration, typically 1 to 6 mol of water per mol of salt. Aluminum chlorohydrate is particularly preferred (i.e. X is Cl in the above formula), and specifically 5/6-basic aluminum chlorohydrate, wherein “a” is 1, such that the molar ratio of aluminum to chlorine is 1.9:1 to 2.1:1.

Preferred activated aluminum zirconium salts are those that are mixtures or complexes of the aluminum salts described above with zirconium salts of the formula ZrO(OH)_{2-pb}Y_{b}, in which Y is Cl, Br, l, NO₃ or SO₄, b is a rational number from 0.8 to 2 and p is the valence of Y, as disclosed in U.S. Pat. No. 6,074,632 for example. The zirconium salts generally likewise contain a little associatively bonded water of hydration, typically 1 to 7 mol of water per mol of salt. The zirconium salt is preferably zirconyl hydroxychloride of the formula ZrO(OH)_{2-b}Cl_{b}, in which b is a rational number from 0.8 to 2, preferably 1.0 to 1.9. Preferred aluminum zirconium salts have a molar Al:Zr molar ratio of 2 to 10 and a metal to (X+Y) ratio of 0.73 to 2.1, preferably 0.9 to 1.5. A particularly preferred salt is aluminum zirconium chlorohydrate (i.e. X and Y are Cl) with a molar Al:Zr ratio of 2 to 10 and a molar metal to Cl ratio of 0.9 to 2.1. The term aluminum zirconium chlorohydrate encompasses the tri-, tetra-, penta- and octachlorohydrate forms.

Zirconium salts that are preferred according to the invention have the general formula ZrO(OH)₂₋ₐClₐ.xH₂O where a=1.5 to 1.87; x=1 to 7, with a and x being rational numbers. These zirconium salts are disclosed for example in the Belgian publication BE 825146.

Further preferred sweat-inhibiting active ingredients are disclosed in U.S. Pat. No. 6,663,854 and US 20040009133.

The sweat-inhibiting active ingredients can be present both in solubilized and in undissolved, suspended form.

If the sweat-inhibiting active ingredients are present suspended in a water-immiscible carrier, it is preferable for reasons of product stability for the active ingredient particles to have a number-average particle size of 0.1 to 200 μm, preferably 1 to 50 μm, particularly preferably 3 to 20 μm and exceptionally preferably 5 to 10 μm. Preferred active ingredient particles have a volume-average particle size of 0.2 to 220 μm, preferably 3 to 60 μm, particularly preferably 4 to 25 μm and exceptionally preferably 10 to 15.5 μm.

Preferred aluminum salts and aluminum zirconium salts have a molar metal to chloride ratio of 0.9 to 2.0, preferably 1.0 to 1.51, particularly preferably 1.1 to 1.5, exceptionally preferably 1.3 to 1.4.

Preferred aluminum zirconium chlorohydrates have the empirical formula AlₙZr(OH)_{[3n+4-m(n+1)]}(Cl)_{[m(n+1)]} where n=2.0 to 10.0, preferably 3.0 to 8.0, m=0.77 to 1.11 (corresponding to a molar metal (Al+Zr) to chloride ratio of 1.3 to 0.9), preferably m=0.91 to 1.11 (corresponding to M:CI=1.1 to 0.9), and particularly preferably m=1.00 to 1.11 (corresponding to M:CI=1.0 to 0.9), furthermore very preferably m=1.02 to 1.11 (corresponding to M:CI=0.98 to 0.9) and very preferably m=1.04 to 1.11 (corresponding to M:CI=0.96 to 0.9).

Further preferred aluminum zirconium trichlorohydrates have the empirical formula Al₄(OH)₁₀Cl₂.Zr(OH)Cl.

Further preferred aluminum zirconium tetrachlorohydrates have the empirical formula Al₄(OH)₁₀Cl₂.ZrCl₂.

Further preferred aluminum zirconium pentachlorohydrates have the empirical formula Al₈(OH)₂₀Cl₆.Zr(OH)Cl.

Further preferred aluminum zirconium octachlorohydrates have the empirical formula Al₈(OH)₁₈Cl₆.Zr(OH)Cl.

These salts generally contain a little associatively bonded water of hydration, typically 1 to 6 mol of water per mol of salt, corresponding to 1 to 30 wt. %, preferably 4 to 13 wt. % of water of hydration.

Preferred sweat-inhibiting salts are aluminum zirconium tetrachlorohydrates (molar ratio Al:Zr=2 to 6; M:Cl=0.9 to 1.5, preferably 0.95:1.3, particularly preferably 1.0:1.1), in particular salts with a molar metal to chloride ratio of 0.9 to 1.1, preferably 0.9 to 1.0.

Further preferred sweat-inhibiting salts are aluminum chlorohydrates with a molar metal to chloride ratio of M:Cl=1.9 to 2.1.

The preferred aluminum zirconium chlorohydrates are conventionally associated with an amino acid to prevent polymerization of the zirconium species during production. Preferred stabilizing amino acids are selected from glycine, alanine, leucine, isoleucine, β-alanine, cysteine, valine, serine, tryptophane, phenylalanine, methionine, β-amino-n-butanoic acid and γ-amino-n-butanoic acid and the salts thereof, in each case in the d form, 1 form or dl form; glycine is particularly preferred. The amino acid is contained in an amount from 1 to 3 mol, preferably 1.3 to 1.8 mol, per mol of zirconium in the salt.

The aforementioned aluminum zirconium trichlorohydrates, aluminum zirconium tetrachlorohydrates, aluminum zirconium pentachlorohydrates and aluminum zirconium octachlorohydrates—both activated and non-activated—are preferably present as a complex with glycine.

Particularly preferred sweat-inhibiting active ingredients are selected from activated aluminum zirconium trichlorohydrex glycines, in particular activated aluminum zirconium trichlorohydrex glycines with an active substance (USP) free from water of crystallization and from glycine of 69.5 to 88 wt. %, preferably 72 to 85 wt. %, particularly preferably 77 to 80 wt. %, relative in each case to the raw material as is, a molar metal:Cl ratio of 0.9 to 1.5 and a molar A1:Zr ratio of 3.4 to 3.8.

Further particularly preferred sweat-inhibiting active ingredients are selected from non-activated aluminum zirconium trichlorohydrex glycines, in particular non-activated aluminum zirconium trichlorohydrex glycines with an active substance (USP) free from water of crystallization and from glycine of 69.5 to 88 wt. %, preferably 72 to 85 wt. %, particularly preferably 77 to 80 wt. %, relative in each case to the raw material as is, a molar metal:Cl ratio of 0.9 to 1.5 and a molar Al:Zr ratio of 3.4 to 3.8.

Further particularly preferred sweat-inhibiting active ingredients are selected from activated aluminum zirconium tetrachlorohydrex glycines, in particular activated aluminum zirconium tetrachlorohydrex glycines with an active substance (USP) free from water of crystallization and from glycine of 72 to 88 wt. %, preferably 77 to 85 wt. %, relative in each case to the raw material as is, a molar metal:Cl ratio of 0.9 to 1.5 and a molar Al:Zr ratio of 3.4 to 3.8.

Further particularly preferred sweat-inhibiting active ingredients are selected from non-activated aluminum zirconium tetrachlorohydrex glycines, in particular non-activated aluminum zirconium tetrachlorohydrex glycines with an active substance (USP) free from water of crystallization and from glycine of 72 to 88 wt. %, preferably 77 to 85 wt. %, relative in each case to the raw material as is, a molar metal:Cl ratio of 0.9 to 1.5 and a molar Al:Zr ratio of 3.4 to 3.8.

Further particularly preferred sweat-inhibiting active ingredients are selected from activated aluminum zirconium pentachlorohydrex glycines, in particular activated aluminum zirconium pentachlorohydrex glycines with an active substance (USP) free from water of crystallization and from glycine of 72 to 88 wt. %, preferably 77 to 86 wt. %, particularly preferably 78 to 81.5 wt. %, relative in each case to the raw material as is, a molar metal:Cl ratio of 1.51 to 2.0 and a molar Al:Zr ratio of 9.2 to 9.8.

Further particularly preferred sweat-inhibiting active ingredients are selected from non-activated aluminum zirconium pentachlorohydrex glycines, in particular non-activated aluminum zirconium pentachlorohydrex glycines with an active substance (USP) free from water of crystallization and from glycine of 72 to 88 wt. %, preferably 77 to 86 wt. %, particularly preferably 78 to 81.5 wt. %, relative in each case to the raw material as is, a molar metal:Cl ratio of 1.51 to 2.0 and a molar Al:Zr ratio of 9.2 to 9.8.

The water of crystallization content in the aforementioned activated and non-activated aluminum zirconium trichlorohydrex glycines, aluminum zirconium tetrachlorohydrex glycines, aluminum zirconium pentachlorohydrex glycines and aluminum zirconium octachlorohydrex glycines is 1.5 to 20 wt. %, preferably 7 to 15 wt. %, relative in each case to the raw material as is.

Further preferred aluminum zirconium trichlorohydrex glycines have the empirical formula [Al₄(OH)₁₀Cl₂.Zr(OH)Cl].NH₂CH₂COOH.

Further preferred aluminum zirconium tetrachlorohydrex glycines have the empirical formula [Al₄(OH)₁₀Cl₂.ZrOCl₂].NH₂CH₂COOH.

Further preferred aluminum zirconium pentachlorohydrex glycines have the empirical formula [Al₈(OH)₂₀Cl₄.Zr(OH)Cl].NH₂CH₂COOH.

Further preferred aluminum zirconium octachlorohydrex glycines have the empirical formula [Al₈(OH)₁₈Cl₆.Zr(OH)Cl].NH₂CH₂COOH or [Al₈(OH)₁₈Cl₆. ZrOCl₂].NH₂CH₂COOH.

Also preferred according to the invention are aluminum zirconium chlorohydrate glycine salts stabilized with betaine ((CH₃)₃N⁺—CH₂—COO⁻). Particularly preferred corresponding compounds have a molar total (betaine+glycine)/Zr ratio of (0.1 to 3.0):1, preferably (0.7 to 1.5):1, and a molar ratio of betaine to glycine of at least 0.001:1. Corresponding compounds are disclosed in U.S. Pat. No. 7,105,691 for example.

In a particularly preferred embodiment according to the invention an “activated” salt is included as a particularly effective antiperspirant salt, in particular one with a high HPLC peak 5 aluminum content, in particular with a peak 5 surface area of at least 33%, particularly preferably at least 45%, relative to the total surface area under peaks 2 to 5, measured by HPLC of a 10 wt. % aqueous solution of the active ingredient under conditions in which the aluminum species is dissolved into at least 4 successive peaks (referred to as peaks 2 to 5). Preferred aluminum zirconium salts having a high HPLC peak 5 aluminum content (also referred to as “E⁵AZCH”) are disclosed in U.S. Pat. No. 6,436,381 and U.S. Pat. No. 6,649,152 for example.

Such activated “E⁵AZCH” salts are also preferred in which the HPLC peak 4 to peak 3 surface area ratio is at least 0.4, preferably at least 0.7, particularly preferably at least 0.9.

Further particularly preferred antiperspirant active ingredients are aluminum zirconium salts having a high HPLC peak 5 aluminum content that are additionally stabilized with a water-soluble strontium salt and/or with a water-soluble calcium salt. Corresponding salts are disclosed in U.S. Pat. No. 6,923,952 for example.

Further preferred antiperspirant active ingredients are selected from astringent titanium salts such as are disclosed in GB 2299506 A for example.

The antiperspirant active ingredients can be used as non-aqueous solutions or as glycolic solubilizates.

Particularly preferred compositions according to the invention have the characterizing feature that the at least one antiperspirant active ingredient is included in an amount from 5 to 40 wt. %, preferably 10 to 35 wt. %, particularly preferably 11 to 28 wt. % and exceptionally preferably 12 to 20 wt. %, relative to the total weight of active substance (USP) free from water of crystallization and from ligands in the complete composition. For sprays containing propellants these specified amounts relate to the weight of the propellant-free composition.

In a particularly preferred embodiment the composition contains an astringent aluminum salt, in particular aluminum chlorohydrate, particularly preferably aluminum chlorohydrate having an active substance (USP) free from water of crystallization of 72 to 88 wt. %, relative to the raw material as is. Preferred non-activated aluminum chlorohydrates are obtainable for example in powder form as Micro Dry®, Micro Dry® Ultrafine or Micro Dry®-323 from Summit Reheis, as Chlorhydrol® (powder) and in activated form as Reach® 101, Reach® 103, Reach® 501 from Reheis/Summit or AACH-7171 from Summit. An aluminum sesquichlorohydrate that is likewise particularly preferred is offered by Reheis under the name Reach® 301.

The use of aluminum zirconium tetrachlorohydrex glycine complexes can also be particularly preferred according to the invention. Aluminum zirconium tetrachlorohydrex glycine complexes are particularly preferred that are commercially available as a powder from Summit Reheis for example under the name Rezal® 36 GP, Summit AZG-369 or Summit AZG-364 or, in an activated grade, as Summit Reach® AZP-908.

Also particularly preferred are aluminum zirconium pentachlorohydrex glycine complexes that are commercially available as a powder from Summit for example in an activated grade under the names AAZG-3108 and AAZG-3110.

The antiperspirant active ingredients are used in the formulations according to the invention in an amount from 1 to 40 wt. %, preferably 3 to 15 wt. %, relative to the total mass of the preparation, i.e. including any propellants that are present.

In stick formulations the proportion of antiperspirant active ingredients is preferably in the range from 10 to 25 wt. %, relative in each case to the total mass of the preparation.

Further particularly preferred stick compositions according to the invention have the characterizing feature that the at least one antiperspirant active ingredient is included in an amount from 5 to 40 wt. %, preferably 10 to 35 wt. %, particularly preferably 11 to 28 wt. % and exceptionally preferably 12 to 20 wt. %, relative to the total weight of active substance (USP) free from water of crystallization and from ligands in the complete composition.

Particularly preferred spray compositions according to the invention have the characterizing feature that the at least one antiperspirant active ingredient is included in an amount from 5 to 40 wt. %, preferably 10 to 35 wt. %, particularly preferably 11 to 28 wt. % and exceptionally preferably 12 to 20 wt. %, relative to the total weight of active substance (USP) free from water of crystallization in the propellant-free complete composition.

Antiperspirant active ingredients from the group of anticholinergics, such as for example 4-[(2-cyclopentyl-2-hydroxyphenylacetyl)oxy]-1,1-dimethyl piperidinium salts, in particular 4-[(2-cyclopentyl-2-hydroxyphenylacetyl)oxy]-1,1-dimethyl piperidinium bromide, can be added in a proportion of preferably 0.05 to 1.0 wt. %, preferably 0.1% to 0.7%, in particular 0.3% to 0.5 wt. %, relative to the total mass of the preparation.

Vicinal diols and similar active ingredients from the group of osmotically active substances can also be added to the preparations according to the invention as an antiperspirant active ingredient, preferably in a proportion of 10 to 50 wt. %, preferably 15% to 30%, in particular 15 to 25 wt. %, relative to the total mass of the preparation.

It is of course also possible to add further antiperspirant active ingredients and/or deodorants.

Deodorant active ingredients that are preferred according to the invention that can be included in addition to the silver salt applied to a carrier are odor absorbers, deodorizing ion exchangers, bacteriostatic agents, prebiotic components and enzyme inhibitors or, particularly preferably, combinations of the cited active ingredients.

Preferred odor absorbers are for example zeolites, zinc ricinoleate, cyclodextrins, certain metal oxides, such as for example aluminum oxide, and chlorophyll. They are preferably used in an amount from 0.1 to 10 wt. %, particularly preferably 0.5 to 7 wt. % and exceptionally preferably 1 to 5 wt. %, relative in each case to the complete composition.

Bacteriostatic or antimicrobial active ingredients are understood according to the invention to be active ingredients that reduce the number of skin bacteria involved in odor formation or inhibit their growth. These bacteria include inter alia various species from the group of Staphylococci, the group of Corynebacteria, Anaerococci and Micrococci.

Organohalogen compounds and halides, quaternary ammonium compounds, a series of plant extracts and zinc compounds are preferred in particular according to the invention as bacteriostatic or antimicrobial active ingredients. These include inter alia triclosan, chlorhexidine and chlorhexidine gluconate, 3,4,4′-trichlorocarbanilide, bromochlorophene, dichlorophen, chlorothymol, chloroxylenol, hexachlorophene, dichloro-m-xylenol, dequalinium chloride, domiphen bromide, ammonium phenol sulfonate, benzalkonium halides, benzalkonium cetyl phosphate, benzalkonium saccharinates, benzethonium chloride, cetyl pyridinium chloride, lauryl pyridinium chloride, lauryl isoquinolinium bromide, methyl benzethonium chloride. Phenol, phenoxyethanol, disodium dihydroxyethyl sulfosuccinyl undecylenate, sodium bicarbonate, zinc lactate, sodium phenol sulfonate and zinc phenol sulfonate, ketoglutaric acid, terpene alcohols such as for example farnesol, chlorophyllin copper complexes, α-monoalkyl glycerol ethers having a branched or linear saturated or unsaturated, optionally hydroxylated C₆-C₂₂ alkyl residue, particularly preferably α-(2-ethylhexyl)glycerol ether, available commercially as Sensiva® SC 50 (from Schülke & Mayr), carboxylic acid esters of mono-, di- and triglycerol (for example glycerol monolaurate, diglycerol monocaprinate), lantibiotics and plant extracts (for example green tea and constituents of linden blossom oil) can also be used.

Further preferred deodorant active ingredients are selected from prebiotic components, which are understood according to the invention to be components that inhibit only or at least predominantly the odor-forming bacteria of the skin microflora but not the desirable, i.e. non-odor-forming bacteria that belong to a healthy skin microflora. These explicitly include conifer extracts, in particular from the group of Pinaceae, and plant extracts from the group of Sapindaceae, Araliaceae, Lamiaceae and Saxifragaceae, in particular extracts of Picea spp., Paullinia sp., Panax sp., Lamium album or Ribes nigrum, and mixtures of these substances.

Further preferred deodorant active ingredients are selected from the bacteriostatic perfume oils and Deosafe® perfume oils available from Symrise, formerly Haarmann & Reimer.

Enzyme inhibitors include substances that inhibit the enzymes responsible for breaking down sweat, in particular arylsulfatase, β-glucuronidase, aminoacylase, esterases, lipases and/or lipoxigenase, for example trialkyl citric acid esters, in particular triethyl citrate, or zinc glycinate.

Preferred deodorant or antiperspirant compositions according to the invention have the characterizing feature that the at least one deodorant active ingredient is selected from arylsulfatase inhibitors, β-glucuronidase inhibitors, aminoacylase inhibitors, esterase inhibitors, lipase inhibitors and lipoxigenase inhibitors, alpha-monoalkyl glycerol ethers having a branched or linear saturated or unsaturated, optionally hydroxylated C₆-C₂₂ alkyl residue, in particular α-(2-ethylhexyl)glycerol ether, phenoxyethanol, bacteriostatic perfume oils, Deosafe® perfume oils (Deosafe® is a registered trademark of Symrise, formerly Haarmann & Reimer), prebiotic components, trialkyl citric acid esters, in particular triethyl citrate, active ingredients that reduce the number of skin bacteria from the group of Staphylococci, Corynebacteria, Anaerococci and Micrococci involved in odor formation or inhibit their growth, zinc compounds, in particular zinc phenol sulfonate and zinc ricinoleate, organohalogen compounds, in particular triclosan, chlorhexidine, chlorhexidine gluconate and benzalkonium halides, quaternary ammonium compounds, in particular cetyl pyridinium chloride, odor absorbers, in particular silicates and zeolites, sodium bicarbonate, lantibiotics, and mixtures of the aforementioned substances.

Further preferred compositions according to the invention have the characterizing feature that the at least one deodorant active ingredient is included in a total amount from 0.1 to 10 wt. %, preferably 0.2 to 7 wt. %, particularly preferably 0.3 to 5 wt. % and exceptionally preferably 0.4 to 1.0 wt. %, relative in each case to the total weight of active substance of the deodorant active ingredient or deodorant active ingredients in the complete composition.

The passivating agents, in particular disteardimonium hectorite or talc, are preferably included in a proportion of 1 to 10 wt. %, preferably in the range from 2 to 7 wt. %, particularly preferably 3 to 5 wt. %, relative to the total mass of the preparation or active ingredient mixture.

The aqueous suspension of at least one silver halide applied to a carrier, including water, is advantageously added in a proportion of a maximum of 5 wt. %, preferably 2 wt. %, in particular 0.1 to 2 wt. %, relative to the total mass of the active ingredient mixture.

Further cosmetic compositions that are preferred according to the invention have the characterizing feature that the aqueous suspension of at least one silver halide applied to a carrier is included in a total amount from 1 to 100 ppm silver ions (Ag⁺), preferably 2 to 50 ppm silver ions, particularly preferably 5 to 20 ppm silver ions, exceptionally preferably 7 to 10 ppm silver ions, relative in each case to the weight of the propellant-free composition according to the invention.

The mixture of active ingredients is the same as the preparation, but if the preparation is an aerosol, the proportions relate to the active mixture excluding propellant.

The preparations according to the invention are preferably applied from standard aerosol and stick packaging means. They are suitable in particular for spraying through standard antiperspirant valves and spray heads, as they help to avoid clogging problems.

The advantage of the preparations according to the invention is therefore that expensive special packaging means such as powder valves for example can be dispensed with. Owing to their special design or geometry, such valves are less sensitive to clogging by larger particles. The disadvantage of the use of such powder valves, however, is that the special shape generally also requires the use of adapted spray heads. This entails additional costs and a diversification of the range of packaging means and hence additional logistics expense. These disadvantages are avoided according to the invention.

A further problem that is solved according to the invention is that when water-soluble encapsulated perfumes that react to the presence of water by swelling, sticking together and disintegrating are used at the same time, these disadvantages are likewise not observed because water is extracted from the perfume. Despite the presence of water (max. 8 wt. % (see above)), any water-soluble capsule materials for perfume oils can thus be combined according to the invention with aqueous suspensions of at least one silver halide applied to a carrier. Modified starch (INCI: Sodium Starch Octenylsuccinate or Modified Starch) can therefore preferably be used as the capsule material.

The amount of perfume capsules used is advantageously in the range from 0.3 to 0.8 wt. %, relative to the total mass of the preparation. Approximately 0.8 to 1.2 wt. % of conventional (free) perfume oil is preferably then additionally included. The perfume capsules are preferably made up of approximately 40 wt. % of sodium starch octenylsuccinate, 10 wt. % of mannitol and approximately 50 wt. % of perfume oil.

The cosmetic and dermatological preparations according to the invention can contain cosmetic auxiliary substances such as are conventionally used in such preparations, for example preservatives, bactericides, UV filters, antioxidants, vitamins, minerals, suspended solids particles, perfumes, substances to prevent foaming, dyes, pigments having a coloring action, thickening agents, moisturizing and/or moisture-retaining substances or other conventional constituents of a cosmetic or dermatological formulation, such as alcohols, polyols, polymers, foam stabilizers or silicone derivatives.

A further preferred embodiment of the invention thus has the characterizing feature that the composition according to the invention or the composition used according to the invention contains at least one monosaccharide having 5 or 6 carbon atoms and/or at least one disaccharide.

A particularly preferred embodiment of the invention has the characterizing feature that the monosaccharide having 5 or 6 carbon atoms is selected from glucose, fructose, galactose, arabinose, ribose, xylose, lyxose, allose, altrose, mannose, gulose, idose, talose, fucose and rhamnose. Of these, glucose, galactose, fructose, fucose and rhamnose are exceptionally preferred, in particular glucose and galactose.

A further particularly preferred embodiment of the invention has the characterizing feature that the disaccharide is selected from sucrose, lactose and trehalose. Of these, sucrose and lactose are exceptionally preferred, in particular lactose.

A further particularly preferred embodiment of the invention has the characterizing feature that at least one monosaccharide having 5 or 6 carbon atoms and/or at least one disaccharide is included in a total amount from 0.001 to 2.0 wt. %, preferably 0.005 to 1.0 wt. %, particularly preferably 0.01 to 0.5 wt. % and exceptionally preferably 0.03 to 0.1 wt. %, relative in each case to the complete composition.

If the compositions according to the invention are present in the form of a stick, they preferably contain a lipid or wax matrix comprising at least one lipid or wax component having a melting point>50° C.

Waxes are generally of a solid to crumbly hard consistency, coarsely to finely crystalline, translucent to opaque, but not glassy, and melt above 50° C. without decomposing. At even a little above the melting point they are of low viscosity and have a highly temperature-dependent consistency and solubility.

Natural vegetable waxes, for example candelilla wax, carnauba wax, Japan wax, sugar cane wax, ouricury wax, cork wax, sunflower wax, fruit waxes such as orange wax, lemon wax, grapefruit wax, and animal waxes, for example beeswax, shellac wax and spermaceti wax, are preferred for example according to the invention. Within the meaning of the invention it can be particularly preferable to use hydrogenated or hardened waxes. Chemically modified waxes, in particular hard waxes such as for example montan ester waxes, hydrogenated jojoba waxes and Sasol waxes, can also be used as the wax component. Synthetic waxes that are likewise preferred according to the invention include for example polyalkylene waxes and polyethylene glycol waxes, C₂₀-C₄₀ diallyl esters of dimeric acids, C₃₀₋₅₀ alkyl beeswax and alkyl and alkylaryl esters of dimeric fatty acids.

A particularly preferred wax component is selected from at least one ester of a saturated, monohydric C₁₆-C₆₀ alcohol and a saturated C₉-C₃₆ monocarboxylic acid. According to the invention these include lactides, the cyclic double esters of α-hydroxycarboxylic acids of the corresponding chain length. Esters of fatty acids and long-chain alcohols have proved particularly advantageous for the composition according to the invention because they impart excellent sensory properties to the antiperspirant preparation and high stability to the stick as a whole. The esters consist of saturated branched or unbranched monocarboxylic acids and saturated branched or unbranched monohydric alcohols. Esters of aromatic carboxylic acids or hydroxycarboxylic acids (e.g. 12-hydroxystearic acid) and saturated branched or unbranched alcohols can also be used according to the invention, provided that the wax component has a melting point>50° C. It is particularly preferable to select the wax component from the group of esters of saturated branched or unbranched alkane carboxylic acids having a chain length of 12 to 24 C atoms and saturated branched or unbranched alcohols having a chain length of 16 to 50° C. atoms that have a melting point of >50° C.

In particular, C₁₆₋₃₆ alkyl stearates and C₁₈₋₃₈ alkylhydroxystearoyl stearates, C₂₀₋₄₀ alkyl erucates and cetearyl behenate can be advantageous as the wax component. The wax or wax components have a melting point>50° C., preferably >60° C.

A particularly preferred embodiment of the invention contains a C₂₀-C₄₀ alkyl stearate as the wax component. This ester is known under the name Kesterwachs® K82H or Kesterwachs® K80H and is sold by Koster Keunen Inc. It is the synthetic simulation of the monoester fraction of beeswax and is distinguished by its hardness, its oil gelling capacity and its broad compatibility with lipid components. This wax can be used as a stabilizer and consistency regulator for W/O and O/W emulsions. The advantage of Kesterwachs is that even at low concentrations it has an excellent oil gelling capacity, so it does not make the stick material too heavy and allows a velvety wear. A further particularly preferred embodiment of the invention contains cetearyl behenate, i.e. mixtures of cetyl behenate and stearyl behenate, as the wax component. This ester is known under the name Kesterwachs® K62 and is sold by Koster Keunen Inc.

Other preferred lipid or wax components having a melting point>50° C. are the triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (tribehenin) or glyceryl tri-12-hydroxystearate, also synthetic full esters of fatty acids and glycols or polyols having 2 to 6 carbon atoms, provided they have a melting point above 50° C., for example preferably C₁₈-C₃₆ acid triglyceride (Syncrowax® HGL-C). Hydrogenated castor oil, obtainable for example as the commercial product Cutina® HR, is particularly preferred according to the invention as the wax component.

Other preferred lipid or wax components having a melting point>50° C. are the saturated linear C₁₄-C₃₆ carboxylic acids, in particular myristic acid, palmitic acid, stearic acid and behenic acid, as well as mixtures of these compounds, for example Syncrowax® AW 1C(C₁₈-C₃₆ fatty acids) or Cutina® FS 45 (palmitic and stearic acid).

Preferred deodorant or antiperspirant sticks according to the invention have the characterizing feature that the lipid or wax component a) is selected from esters of a saturated, monohydric C₁₆-C₆₀ alkanol and a saturated C₈-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which can be hydroxylated, candelilla wax, carnauba wax, beeswax, saturated linear C₁₄-C₃₆ carboxylic acids and mixtures of the aforementioned substances. Particularly preferred lipid or wax component mixtures a) are selected from mixtures of cetyl behenate, stearyl behenate, hydrogenated castor oil, palmitic acid and stearic acid. Further particularly preferred lipid or wax component mixtures a) are selected from mixtures of C₂₀-C₄₀ alkyl stearate, hydrogenated castor oil, palmitic acid and stearic acid.

Further preferred deodorant or antiperspirant sticks according to the invention have the characterizing feature that the lipid or wax component(s) is/are included in total in amounts from 4 to 20 wt. %, preferably 8 to 15 wt. %, relative to the complete composition. In a particularly preferred embodiment the ester(s) of a saturated, monohydric C₁₆-C₆₀ alcohol and a saturated C₈-C₃₆ monocarboxylic acid, representing the lipid or wax component(s), is/are included in amounts from in total 2 to 10 wt. %, preferably 2 to 6 wt. %, relative to the complete composition.

Preferred compositions according to the invention in the form of a spray or stick preferably also contain at least one oil that is liquid at 20° C. and that is neither a fragrance component nor an essential oil. Oils that are preferred according to the invention are selected from branched saturated or unsaturated fatty alcohols having 6 to 30 carbon atoms. These alcohols are frequently also referred to as Guerbet alcohols, as they are obtainable by the Guerbet reaction. Preferred alcohol oils are hexyl decanol (Eutanol® G 16), octyl dodecanol (Eutanol® G) and 2-ethylhexyl alcohol. Further preferred oil components are mixtures of Guerbet alcohols and Guerbet alcohol esters, for example the commercial product Cetiol® PGL (hexyl decanol and hexyldecyl laurate).

Further oils that are preferred according to the invention are selected from the triglycerides of linear or branched, saturated or unsaturated, optionally hydroxylated C₈₋₃₀ fatty acids. The use of natural oils, for example soybean oil, cottonseed oil, sunflower oil, palm oil, palm kernel oil, linseed oil, almond oil, castor oil, corn oil, olive oil, rapeseed oil, sesame oil, thistle oil, wheat germ oil, peach kernel oil and the liquid components of coconut butter and the like, can be particularly suitable. Synthetic triglyceride oils are also suitable, however, in particular capric/caprylic triglycerides, for example the commercial products Myritol® 318, Myritol® 331 (Cognis) or Miglyol® 812 (Hills) with unbranched fatty acid esters as well as glyceryl triisostearin and the commercial products Estol® GTEH 3609 (Uniqema) or Myritol® GTEH (Cognis) with branched fatty acid esters.

Further oils that are particularly preferred according to the invention are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further oils that are particularly preferred according to the invention are selected from the addition products of 1 to 5 propylene oxide units with mono- or polyhydric C₈₋₂₂ alkanols such as octanol, decanol, decanediol, lauryl alcohol, myristyl alcohol and stearyl alcohol, for example PPG-2-myristyl ether and PPG-3-myristyl ether (Witconol® APM).

Further oil components that are preferred according to the invention are selected from the esters of linear or branched saturated or unsaturated fatty alcohols having 2 to 30 carbon atoms with linear or branched saturated or unsaturated fatty acids having 2 to 30 carbon atoms, which can be hydroxylated. These include hexyldecyl stearate (Eutanol® G 16 S), hexyldecyl laurate, isodecyl neopentanoate, isononyl isononanoate, 2-ethylhexyl palmitate (Cegesoft® C 24) and 2-ethylhexyl stearate (Cetiol® 868). Likewise suitable within limits are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and dipalmitate.

Further oil components that are preferred according to the invention are selected from the addition products of at least 6 ethylene oxide and/or propylene oxide units with mono- or polyhydric C₃₋₂₂ alkanols such as butanol, butanediol, myristyl alcohol and stearyl alcohol, for example PPG-14 butyl ether (Ucon Fluid® AP), PPG-9 butyl ether (Breox® B25), PPG-10 butanediol (Macol® 57) and PPG-15 stearyl ether (Arlamol® E).

Further oil components that are preferred according to the invention are selected from the C₈-C₂₂ fatty alcohol esters of monobasic or polybasic C₂-C₇ hydroxycarboxylic acids, in particular the esters of glycolic acid, lactic acid, malic acid, tartaric acid, citric acid and salicylic acid. Such esters based on linear C_{14/15} alkanols, for example C₁₂-C₁₅ alkyl lactate, and on C_{12/13} alkanols branched in 2-position are available under the trademark Cosmacol® from Nordmann, Rassmann GmbH & Co, Hamburg, in particular the commercial products Cosmacol® ESI, Cosmacol® EMI and Cosmacol® ETI.

Further oil components that are preferred according to the invention are selected from the symmetrical, asymmetrical or cyclic esters of carbonic acid with fatty alcohols, for example glycerol carbonate, dicaprylyl carbonate (Cetiol® CC) or the esters of DE 197 56 454 A1.

Further oil components that are preferred according to the invention are selected from the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols.

It can be exceptionally preferable according to the invention to use mixtures of the aforementioned oils.

Further oil components that are preferred according to the invention are selected from silicone oils and hydrocarbon oils.

Silicone oils that are preferred according to the invention are selected from dialkyl and alkylaryl siloxanes, such as for example cyclopentasiloxane, cyclohexasiloxane, dimethylpolysiloxane and methylphenylpolysiloxane, but also including hexamethyldisiloxane, octamethyltrisiloxane and decamethyltetrasiloxane. Further silicone oils that are preferred according to the invention are selected from volatile silicone oils, which can be cyclic, such as for example octamethyl cyclotetrasiloxane, decamethyl cyclopentasiloxane and dodecamethyl cyclohexasiloxane and mixtures thereof, such as are contained for example in the commercial products DC 244, 245, 344 and 345 from Dow Corning, or linear, for example hexamethyl disiloxane (L₂), octamethyl trisiloxane (L₃), decamethyl tetrasiloxane (L₄), any two-component and three-component mixtures of L₂, L₃ and/or L₄, such as are contained for example in the commercial products DC 2-1184, Dow Corning® 200 (0.65 cSt) and Dow Corning® 200 (1.5 cSt) from Dow Corning. Further silicone oils that are preferred according to the invention are selected from non-volatile higher-molecular-weight linear dimethyl polysiloxanes, available commercially for example under the name Dow Corning® 190, Dow Corning® 200 Fluid with viscosities in the range from 5 to 100 cSt, preferably 5 to 50 cSt or 5 to 10 cSt, and Baysilon® 350 M.

Natural and synthetic hydrocarbons that are preferred according to the invention are selected from paraffin oils, isohexadecane, isoeicosane, polyisobutenes and polydecenes, which are available for example under the name Emery® 3004, 3006, 3010 or under the name Ethylflo® from Albemarle or Nexbase® 2004G from Nestle, as well as 1,3-di-(2-ethylhexyl)cyclohexane (Cetiol® S).

Particularly preferred deodorant or antiperspirant compositions according to the invention have the characterizing feature that the oil(s) that is/are liquid at 20° C. is/are included in a total amount from 0.1 to 80 wt. %, preferably 2 to 20 wt. %, particularly preferably 3 to 15 wt. %, relative in each case to the total weight of the composition.

In a further preferred embodiment of the invention a proportion of the oil components of at least 80 wt. % has a refractive index n_{D} of 1.39 to 1.51. It is particularly preferable for 5 to 40 to 50 wt. %, exceptionally preferably 10 to 12 to 25 to 30 wt. % of the oil components to have a refractive index n_{D} of 1.43 to 1.51, preferably 1.44 to 1.49, particularly preferably 1.45 to 1.47 to 1.485, at 20° C. (measured at λ=589 nm).

Further preferred compositions according to the invention have the characterizing feature that at least one wax component having a melting point in the range from 25 to 50° C. is included, selected from coconut fatty acid glycerol mono-, di- and triesters, Butyrospermum parkii (shea butter) and esters of saturated, monohydric C₈-C₁₈ alcohols with saturated C₁₂-C₁₈ monocarboxylic acids and mixtures of these substances. These low-melting-point wax components make it possible to optimize the consistency of the product and minimize visible residues on the skin. Commercial products with the INCI name Cocoglycerides, particularly preferably a mixture of C₁₂-C₁₈ mono-, di- and triglycerides that melts in the range from 30 to 32° C., such as is available for example under the commercial name Novata® AB from Cognis, and the products from the Softisan range (Sasol Germany GmbH) with the INCI name Hydrogenated Cocoglycerides, in particular Softisan 100, 133, 134, 138, 142, are particularly preferred. Further preferred esters of saturated, monohydric C₁₂-C₁₈ alcohols with saturated C₁₂-C₁₈ monocarboxylic acids are stearyl laurate, cetearyl stearate (e.g. Crodamol® CSS), stearyl stearate (e.g. Estol 3706), cetyl palmitate (e.g. Cutina® CP, melting point: 46 to 50° C.) and myristyl myristate (e.g. Cetiol® MM, melting point: 38 to 42° C.).

Further preferred stick compositions according to the invention have the characterizing feature that at least one wax component having a melting point in the range from 25 to 50° C. that is not a fatty alcohol is included in a total amount from 0.01 to 10 wt. %, preferably 0.5 to 8 wt. %, particularly preferably 1 to 5 wt. % and exceptionally preferably 1.1 to 4 wt. %, relative to the stick composition.

Further particularly preferred deodorant or antiperspirant compositions according to the invention have the characterizing feature that at least one lipid or wax component having a melting point in the range from 25 to <50° C. is included in amounts from 0.01 to 20 wt. %, preferably 3 to 20 wt. %, particularly preferably 5 to 18 wt. % and exceptionally preferably 6 to 15 wt. %, relative to the complete composition.

Further preferred deodorant or antiperspirant sticks according to the invention have the characterizing feature that the lipid or wax component(s) is/are included in total in amounts from 4 to 30 wt. %, preferably 8 to 28 wt. %, particularly preferably 15 to 25 wt. %, exceptionally 20 to 23 wt. %, relative to the stick composition.

Anhydrous antiperspirant wax sticks contain approximately 40 to 60 wt. % of at least one cosmetic oil that is liquid under normal conditions, approximately 15 to 28 wt. % of a fat component that is solid under normal conditions, approximately 80 to 85 wt % of which conventionally has a melting point of approximately 50° C. or conventionally consists of fatty alcohols, in particular stearyl alcohol, but also cetyl alcohol and optionally also arachidyl alcohol and/or behenyl alcohol, while a smaller proportion, approximately 0.5 to 5 wt. %, consists of at least one fat component having a melting point of approximately 55 to 120° C. Moreover, 0.5 to 5 wt. % of at least one fat component having a melting point of approximately 25 to 35° C. can be included. Furthermore, 0.5 to 10 wt. % of at least one filler other than phyllosilicates and talc can be included, which is typically selected from cellulose powders, starches and starch derivatives. Furthermore, 0.1 to 10 wt. %, preferably 1 to 5 wt. %, particularly preferably 2 to 4 wt. % of at least one non-silicone-based oil-in-water emulsifier can be included.

In a particularly preferred embodiment according to the invention the anhydrous antiperspirant wax stick is in the form of a multi-phase stick, in particular a two-phase stick. According to the invention this is understood to mean sticks that contain for example a first wax stick phase as a core and at least a second wax stick phase as a ring around the first phase. In addition to a concentric, annular arrangement of the individual phases, other arrangements are also possible, in particular an arrangement in strip form. The individual phases can be distinguished from one another by different colors, for example, but also by different constituents. Corresponding multi-phase sticks are disclosed in U.S. Pat. No. 6,936,242 and WO 00/67712 A1 for example. Preferred production methods for such sticks are disclosed in U.S. Pat. No. 6,838,032.

An embodiment as a multi-phase stick, in particular as a two-phase stick, in which only one of the phases contains a specific active ingredient, is particularly preferred.

An embodiment as an anhydrous antiperspirant wax stick that is preferred according to the invention has the characterizing feature that it contains:

5 to 40 wt. %, preferably 10 to 35 wt. %, particularly preferably 11 to 28 wt. % and exceptionally preferably 12 to 20 wt. % (USP) of at least one antiperspirant active ingredient,

30 to 70 wt % of at least one cosmetic oil that is liquid under normal conditions,

15 to 32 wt. % of a fat component that is solid under normal conditions, of which more than 65 wt. %, preferably more than 70 wt. %, particularly preferably more than 80 wt. %, relative in each case to the total content of fat components that are solid under normal conditions, consists of C₁₆-C₃₀ fatty alcohols, preferably selected from the group consisting of stearyl alcohol, cetyl alcohol and mixtures thereof, wherein in addition to stearyl alcohol and/or cetyl alcohol a further 0.1 to 3 wt. %, relative to the complete stick, of arachidyl alcohol and/or behenyl alcohol and/or at least one C₂₄-C₃₀ fatty alcohol is/are particularly preferably included,

furthermore 0.5 to 5 wt. %, relative to the complete stick, of at least one fat component having a melting point of 75 to 120° C. and 0.5 to 8 wt. %, relative to the complete stick, of at least one fat component having a melting point of 25 to 45° C.,

furthermore 0.5 to 30 wt. %, preferably 1 to 25 wt. %, particularly preferably 5 to 20 wt. %, exceptionally preferably 10 to 15 wt. % of talc.

Preferred compositions according to the invention in the form of a spray or stick preferably also contain at least one oil that is liquid at 20° C. and that is neither a fragrance component nor an essential oil.

The compositions according to the invention contain at least one oil as a carrier fluid.

Antiperspirant compositions that are preferred according to the invention contain 30 to 95 wt. %, preferably 40 to 93 wt. %, particularly preferably 50 to 90 wt. %, exceptionally preferably 55 to 85 wt. %, relative in each case to the complete composition, of at least one cosmetic oil that is liquid under normal conditions. A total amount of cosmetic oils that are liquid under normal conditions of 60, 63, 65, 68, 70, 73, 75, 78 or 80 wt. %, relative in each case to the complete composition, can also be particularly preferred according to the invention, with a total amount from 65 to 73 wt. % being particularly preferred.

A distinction is made in cosmetic oils between volatile and non-volatile oils. Non-volatile oils are understood to be oils that at 20° C. and under an ambient pressure of 1013 hPa have a vapor pressure of less than 2.66 Pa (0.02 mm Hg). Volatile oils are understood to be oils that at 20° C. and under an ambient pressure of 1013 hPa have a vapor pressure of 2.66 Pa to 40,000 Pa (0.02 mm to 300 mm Hg), preferably 13 to 12,000 Pa (0.1 to 90 mm Hg), particularly preferably 15 to 8000 Pa, exceptionally preferably 300 to 3000 Pa.

Cosmetic oils that are preferred according to the invention are selected from silicone oils, which include for example dialkyl and alkylaryl siloxanes, such as for example cyclopentasiloxane, cyclohexasiloxane, dimethyl polysiloxane, low-molecular-weight phenyl trimethicone and methylphenyl polysiloxane, but also hexamethyl disiloxane, octamethyl trisiloxane and decamethyl tetrasiloxane. Volatile silicone oils are particularly preferred, which can be cyclic, such as for example octamethyl cyclotetrasiloxane, decamethyl cyclopentasiloxane and dodecamethyl cyclohexasiloxane and mixtures thereof, such as are included for example in the commercial products DC 244, 245, 344 and 345 from Dow Corning (vapor pressure at 20° C. approx. 13 to 15 Pa). Volatile linear silicone oils having 2 to 10 siloxane units are likewise particularly preferred, in particular hexamethyl disiloxane (L₂), octamethyl trisiloxane (L₃), decamethyl tetrasiloxane (L₄) and any two-component and three-component mixtures of L₂, L₃ and/or L₄, preferably mixtures such as are contained for example in the commercial products Dow Corning® 2-1184, Dow Corning® 200 (0.65 cSt) and Dow Corning® 200 (1.5 cSt) from Dow Corning. A further preferred volatile silicone oil is a low-molecular-weight phenyl trimethicone with a vapor pressure at 20° C. of approximately 2000 Pa, such as is available for example from GE Bayer Silicones/Momentive under the name Baysilone Fluid PD 5.

Volatile silicone oils are outstandingly suitable carrier oils for antiperspirant compositions that are preferred according to the invention because they give them a pleasant feel on the skin and reduce staining of clothing. Antiperspirant compositions that are particularly preferred according to the invention are therefore distinguished by a content of at least one volatile silicone oil of 30 to 95 wt. %, preferably 40 to 93 wt. %, particularly preferably 50 to 90 wt. %, exceptionally preferably 55 to 85 wt. %, relative in each case to the complete composition.

In addition to or in place of the at least one volatile silicone oil, at least one volatile non-silicone oil can also be included. Preferred volatile non-silicone oils are selected from C₈-C₁₆ isoparaffins, in particular from isodecane, isoundecane, isododecane, isotridecane, isotetradecane, isopentadecane and isohexadecane, as well as mixtures thereof. C₁₀-C₁₃ isoparaffin mixtures are preferred, in particular those having a vapor pressure at 20° C. of approximately 300 to 400 Pa, preferably 360 Pa. This at least one volatile non-silicone oil too is preferably included in a total amount from 30 to 95 wt. %, preferably 40 to 93 wt. %, particularly preferably 50 to 90 wt. %, exceptionally preferably 55 to 85 wt. %, relative in each case to the complete composition.

Owing to the drier skin feel and faster release of active ingredients, volatile silicone oils, isoparaffins, in particular isodecane, isoundecane, isododecane, isotridecane, isotetradecane, isopentadecane, isohexadecane and isoeicosane, as well as mixtures of volatile silicone oils and isoparaffins, in particular isododecane, isohexadecane or isoeicosane, are particularly preferred as the carrier oil.

Compositions that are preferred according to the invention have the characterizing feature that the at least one carrier oil that is liquid under normal conditions encompasses at least one isoparaffin oil, in particular isodecane, isoundecane, isododecane, isotridecane, isotetradecane, isopentadecane, isohexadecane and isoeicosane.

Further compositions that are preferred according to the invention have the characterizing feature that the carrier oil that is liquid under normal conditions encompasses a mixture of i) a volatile silicone oil selected from cyclomethicone and linear polydimethyl siloxanes having 2 to 10 siloxane units, and ii) at least one isoparaffin oil, isodecane, isoundecane, isododecane, isotridecane, isotetradecane, isopentadecane, isohexadecane and isoeicosane.

In addition to the aforementioned substances conventionally referred to as “volatile” silicone oils and in addition to the aforementioned volatile non-silicone oils, antiperspirant compositions that are particularly preferred according to the invention can also contain at least one non-volatile cosmetic oil selected from non-volatile silicone oils and non-volatile non-silicone oils. The at least one non-volatile oil balances out the negative effect of the volatile oil on the residue performance of antiperspirant compositions that are preferred according to the invention. Due to the relatively fast evaporation of the volatile oils, solid, insoluble constituents, in particular the antiperspirant active ingredients, can become visible on the skin as an unattractive residue. These residues can be successfully masked with a non-volatile oil. Furthermore, with a mixture of non-volatile and volatile oil, parameters such as skin feel, residue visibility and suspension stability can be fine-tuned and better adjusted to the requirements of consumers.

It is of course likewise possible to formulate anhydrous antiperspirant compositions with a small proportion of volatile oils or even without volatile oils.

Preferred non-volatile silicone oils are selected from higher-molecular-weight linear dimethyl polysiloxanes, available commercially for example under the name Dow Corning® 190, Dow Corning® 200 Fluid with kinematic viscosities (25° C.) in the range from 5 to 100 cSt, preferably 6 to 50 cSt or 5 to 10 cSt, and Baysilon® 350 M (with a kinematic viscosity (25° C.) of approximately 350 cSt). Silicone oils that are likewise preferred according to the invention are selected from silicones of the formula (Sil-1), in which x is selected from whole numbers from 1 to 20.

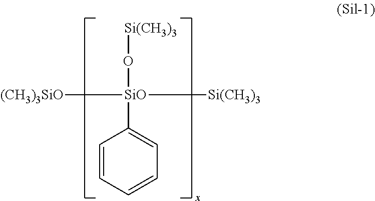

A preferred silicone oil of the formula (Sil-1) is available in various grades, viscosities and volatilities under the INCI name Phenyl Trimethicone. A non-volatile phenyl trimethicone is available for example from Dow Corning under the name Dow Corning 556.

Natural and synthetic hydrocarbons, such as for example paraffin oils, C₁₈-C₃₀ isoparaffins, in particular isoeicosane, polyisobutenes or polydecenes, which are available for example under the name Emery® 3004, 3006, 3010 or under the name Ethylflo® from Albemarle or Nexbase® 2004G from Nestle, as well as 1,3-di-(2-ethylhexyl)cyclohexane (available for example under the trade name Cetiol® S from Cognis), likewise belong to the non-volatile non-silicone oils that are preferred according to the invention.

Further non-volatile non-silicone oils that are preferred according to the invention are selected from the benzoic acid esters of linear or branched C₈₋₂₂ alkanols. Benzoic acid C₁₂-C₁₅ alkyl esters, available for example as the commercial product Finsolv® TN, benzoic acid isostearyl esters, available for example as the commercial product Finsolv® SB, ethylhexyl benzoate, available for example as the commercial product Finsolv® EB, and benzoic acid octyl dodecyl esters, available for example as the commercial product Finsolv® BOD, are particularly preferred. Such benzoic acid ester oils are particularly suitable for masking residues of antiperspirant active ingredients as their refractive index is very close to that of the particularly effective aluminum zirconium mixed salts.

Further non-volatile non-silicone oils that are preferred according to the invention are selected from branched saturated or unsaturated fatty alcohols having 6 to 30 carbon atoms. These alcohols are frequently also referred to as Guerbet alcohols, as they are obtainable by the Guerbet reaction. Preferred alcohol oils are hexyl decanol (Eutanol® G 16), octyl dodecanol (Eutanol® G) and 2-ethylhexyl alcohol.

Further preferred non-volatile non-silicone oils are selected from mixtures of Guerbet alcohols and Guerbet alcohol esters, for example the commercial product Cetiol® PGL (hexyldecanol and hexyldecyl laurate).

Further non-volatile non-silicone oils that are preferred according to the invention are selected from the triglycerides of linear or branched, saturated or unsaturated, optionally hydroxylated C₈₋₃₀ fatty acids. The use of natural oils, for example soybean oil, cottonseed oil, sunflower oil, palm oil, palm kernel oil, linseed oil, almond oil, castor oil, corn oil, rapeseed oil, olive oil, sesame oil, thistle oil, wheat germ oil, peach kernel oil and the liquid components of coconut butter and the like, can be particularly suitable. Synthetic triglyceride oils are also suitable, however, in particular capric/caprylic triglycerides, for example the commercial products Myritol® 318, Myritol® 331 (Cognis) or Miglyol® 812 (Hills) with unbranched fatty acid esters as well as glyceryl triisostearin and the commercial products Estol® GTEH 3609 (Uniqema) or Myritol® GTEH (Cognis) with branched fatty acid esters.

Further non-volatile non-silicone oils that are particularly preferred according to the invention are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further non-volatile non-silicone oils that are particularly preferred according to the invention are selected from the esters of linear or branched saturated or non-volatile non-silicone oils of unsaturated fatty alcohols having 2 to 30 carbon atoms with linear or branched saturated or unsaturated fatty acids having 2 to 30 carbon atoms, which can be hydroxylated. These include isopropyl myristate, isopropyl palmitate, isopropyl isostearate, hexyldecyl stearate (Eutanol® G 16 S), hexyldecyl laurate, isononyl isononanoate, 2-ethylhexyl palmitate (Cegesoft® C 24) and 2-ethylhexyl stearate (Cetiol® 868). Likewise preferred are isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid-2-butyl octanoate, diisotridecyl acetate, n-hexyl laurate, n-decyl oleate, oleyl oleate, oleyl erucate, erucyl oleate and erucyl erucate.

Further non-volatile non-silicone oils that are particularly preferred according to the invention are selected from the addition products of 1 to 5 propylene oxide units with mono- or polyhydric C₈₋₂₂ alkanols such as octanol, decanol, decanediol, lauryl alcohol, myristyl alcohol and stearyl alcohol, for example PPG-2 myristyl ether and PPG-3 myristyl ether (Witconol® APM).

Further non-volatile non-silicone oils that are particularly preferred according to the invention are selected from the addition products of at least 6 ethylene oxide and/or propylene oxide units with mono- or polyhydric C₃₋₂₂ alkanols such as glycerol, butanol, butanediol, myristyl alcohol and stearyl alcohol, which can be esterified if desired, for example PPG-14 butyl ether (Ucon Fluid® AP), PPG-9 butyl ether (Breox® B25), PPG-10 butanediol (Macol® 57), PPG-15 stearyl ether (Arlamol® E) and Glycereth-7 diisononanoate.

Further non-volatile non-silicone oils that are particularly preferred according to the invention are selected from the symmetrical, asymmetrical or cyclic esters of carbonic acid with fatty alcohols, for example glycerol carbonate, dicaprylyl carbonate (Cetiol® CC) or the esters according to the teaching of DE 19756454 A1.

Further oils that can be preferred according to the invention are selected from the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols.

It can be preferable according to the invention to use mixtures of the aforementioned oils.

Preferred compositions according to the invention have the characterizing feature that the carrier oil that is liquid under normal conditions is selected from volatile silicone oils, non-volatile silicone oils, volatile hydrocarbon oils, branched saturated or unsaturated fatty alcohols having 6 to 30 carbon atoms, triglycerides of linear or branched, saturated or unsaturated, optionally hydroxylated C₈-C₃₀ fatty acids, dicarboxylic acid esters of linear or branched C₂-C₁₀ alkanols, esters of branched saturated or unsaturated fatty alcohols having 2 to 30 carbon atoms with linear or branched saturated or unsaturated fatty acids having 2 to 30 carbon atoms, which can be hydroxylated, addition products of 1 to 5 propylene oxide units with mono- or polyhydric C₈₋₂₂ alkanols, addition products of at least 6 ethylene oxide and/or propylene oxide units with mono- or polyhydric C₃₋₂₂ alkanols, C₈-C₂₂ fatty alcohol esters of monobasic or polybasic C₂-C₇ hydroxycarboxylic acids, symmetrical, asymmetrical or cyclic esters of carbonic acid with fatty alcohols, the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols, as well as mixtures of the aforementioned substances.

It can be exceptionally preferable according to the invention to use mixtures of the aforementioned oils to achieve an optimum fine-tuning of the product properties, in particular the residue performance, the skin feel or the release of active ingredients.

Particularly preferred compositions according to the invention have the characterizing feature that the carrier oil that is liquid under normal conditions is a mixture of at least one volatile silicone oil and at least one oil of another type. These are preferably mixtures of at least one volatile silicone oil and at least one non-silicone oil, selected from volatile hydrocarbon oils, branched saturated or unsaturated fatty alcohols having 6 to 30 carbon atoms, triglycerides of linear or branched, saturated or unsaturated, optionally hydroxylated C₈-C₃₀ fatty acids, dicarboxylic acid esters of linear or branched C₂-C₁₀ alkanols, esters of branched saturated or unsaturated fatty alcohols having 2 to 30 carbon atoms with linear or branched saturated or unsaturated fatty acids having 2 to 30 carbon atoms, which can be hydroxylated, addition products of 1 to 5 propylene oxide units with mono- or polyhydric C₈₋₂₂ alkanols, addition products of at least 6 ethylene oxide and/or propylene oxide units with mono- or polyhydric C₃₋₂₂ alkanols, C₈-C₂₂ fatty alcohol esters of monobasic or polybasic C₂-C₇ hydroxycarboxylic acids, symmetrical, asymmetrical or cyclic esters of carbonic acid with fatty alcohols, the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols, as well as mixtures of the aforementioned substances.

Further oil component types that are particularly preferred according to the invention are isoparaffin oils, in particular isododecane, isohexadecane and isoeicosane. Isododecane, isohexadecane and isoeicosane are volatile oil components. As they evaporate relatively quickly after being applied to the skin, the hydrophobic loading of the sweat-inhibiting active ingredient particles is reduced.

Such volatile oil components thus support the release of the sweat-inhibiting active ingredient.

According to a further, likewise preferred embodiment the anhydrous antiperspirant compositions according to the invention contain a small proportion comprising a maximum of 2 wt. %, preferably a maximum of 1 wt. % of cyclomethicone or are even free from cyclomethicone. C₈-C₁₆ isoparaffins, selected in particular from isononane, isodecane, isoundecane, isododecane, isotridecane, isotetradecane, isopentadecane and isohexadecane, as well as mixtures thereof, are particularly preferred as a substitute for cyclomethicone. C₁₀-C₁₃ isoparaffin mixtures are preferred, in particular those having a vapor pressure at 20° C. of approximately 300 to 400 Pa, preferably 360 Pa.

In addition to the at least one aforementioned C₈-C₁₆ isoparaffin, further antiperspirant compositions that are preferred according to the invention contain in addition to 0 to a maximum of 2 wt. %, preferably a maximum of 1 wt. % of cyclomethicone, at least one non-volatile cosmetic oil selected from non-volatile silicone oils and non-volatile non-silicone oils. The at least one non-volatile oil balances out the negative effect of the volatile isoparaffin on the residue performance of antiperspirant compositions that are preferred according to the invention. Due to the relatively fast evaporation of the volatile oils, solid, insoluble constituents, in particular the antiperspirant active ingredients, can become visible on the skin as an unattractive residue. These residues can be successfully masked with a non-volatile oil. Furthermore, with a mixture of non-volatile and volatile oil, parameters such as skin feel, residue visibility and suspension stability can be fine-tuned and better adjusted to the requirements of consumers. Particularly preferred non-volatile oils that are suitable for this purpose are in particular the ester oils 2-ethylhexyl palmitate (e.g. Cegesoft® C 24), hexyldecyl laurate, 2-ethylhexyl stearate, isopropyl myristate, isopropyl palmitate and 2-ethylhexyl laurate, the benzoic acid esters of linear or branched C₈₋₂₂ alkanols, in particular the commercial product Finsolv® TN(C₁₂-C₁₅ alkyl benzoate), C₁₂-C₁₅ alkyl lactate and di-C₁₂-C₁₃ alkyl malate. It can furthermore be particularly preferable to formulate anhydrous antiperspirant compositions according to the invention without cyclomethicone and without volatile linear silicone oils. For this purpose too the ester oils 2-ethylhexyl palmitate (e.g. Cegesoft® C 24), hexyldecyl laurate, 2-ethylhexyl stearate, isopropyl myristate, isopropyl palmitate and 2-ethylhexyl laurate, the benzoic acid esters of linear or branched C₈₋₂₂ alkanols, in particular the commercial product Finsolv® TN (C₁₂-C₁₅ alkyl benzoate), C₁₂-C₁₅ alkyl lactate and di-C₁₂-C₁₃ alkyl malate are particularly preferably suitable.

Oil mixtures that are particularly preferred according to the invention containing 0 to a maximum of 2 wt. %, preferably 0 to a maximum of 1 wt. % of cyclomethicone are 2-ethylhexyl palmitate/isodecane/isoundecane/isododecane/isotridecane, hexyldecyl laurate/isodecane/isoundecane/isododecane/isotridecane, 2-ethylhexyl stearate/isodecane/isoundecane/isododecane/isotridecane, isopropyl myristate/isodecane/isoundecane/isododecane/isotridecane, isopropyl palmitate/isononane/i so decane/isoundecane/iso do decane/isotridecan e, 2-ethylhexyl laurate/isodecane/isoundecane/isododecane/isotridecane, C₁₂-C₁₅ alkyl lactate/isodecane/is oundecane/isododecane/isotridecane, C₁₂-C₁₅ alkyl benzoate/isodecane/isoundecane/isododecane/isotridecane and di-C₁₂-C₁₃ alkyl malate/isodecane/isoundecane/isododecane/isotridecane.

In preferred oil mixtures containing 0 to a maximum of 2 wt. %, preferably 0 to a maximum of 1 wt. % of cyclomethicone, the two oil types (ester/C₈-C₁₆ isoparaffin) are included in equal percentages by weight. Further preferred weight ratios of ester to C₈₋₁₆ isoparaffin are (0.6 to 1):(1 to 3). Further preferred weight ratios of ester to C₈-C₁₆ isoparaffin are 1:(1 to 1.5). Further preferred weight ratios of ester to C₈-C₁₆ isoparaffin are (0.6 to 0.9):(2.5 to 3), in particular 0.8:3.

In further preferred oil mixtures containing 0 to a maximum of 2 wt. %, preferably 0 to a maximum of 1 wt. % of cyclomethicone, the two oil types isopropyl myristate/C₈-C₁₆ isoparaffin are included in equal percentages by weight. Further preferred weight ratios of isopropyl myristate to C₈₋₁₆ isoparaffin are (0.6 to 1):(1 to 3). Further preferred weight ratios of isopropyl myristate to C₈-C₁₆ isoparaffin are 1:(1 to 1.5). Further preferred weight ratios of isopropyl myristate to C₈-C₁₆ isoparaffin are (0.6 to 0.9):(2.5 to 3), in particular 0.8:3.

In further preferred oil mixtures containing 0 to a maximum of 2 wt. %, preferably 0 to a maximum of 1 wt. % of cyclomethicone, the two oil types isopropyl palmitate/C₈-C₁₆ isoparaffin are included in equal percentages by weight. Further preferred weight ratios of isopropyl palmitate to C₈₋₁₆ isoparaffin are (0.6 to 1):(1 to 3). Further preferred weight ratios of isopropyl palmitate to C₈-C₁₆ isoparaffin are 1:(1 to 1.5). Further preferred weight ratios of isopropyl palmitate to C₈-C₁₆ isoparaffin are (0.6 to 0.9):(2.5 to 3), in particular 0.8:3.

In further preferred oil mixtures containing 0 to a maximum of 2 wt. %, preferably 0 to a maximum of 1 wt. % of cyclomethicone, the two oil types 2-ethylhexyl palmitate/C₈-C₁₆ isoparaffin are included in equal percentages by weight. Further preferred weight ratios of 2-ethylhexyl palmitate to C₈₋₁₆ isoparaffin are (0.6 to 1):(1 to 3). Further preferred weight ratios of 2-ethylhexyl palmitate to C₈-C₁₆ isoparaffin are 1:(1 to 1.5). Further preferred weight ratios of 2-ethylhexyl palmitate to C₈-C₁₆ isoparaffin are (0.6 to 0.9):(2.5 to 3), in particular 0.8:3.

In further preferred oil mixtures containing 0 to a maximum of 2 wt. %, preferably 0 to a maximum of 1 wt. % of cyclomethicone, the two oil types C₁₂-C₁₅ alkyl benzoate/C₈-C₁₆ isoparaffin are included in equal percentages by weight. Further preferred weight ratios of C₁₂-C₁₅ alkyl benzoate to C₈₋₁₆ isoparaffin are (0.6 to 1):(1 to 3). Further preferred weight ratios of C₁₂-C₁₅ alkyl benzoate to C₈-C₁₆ isoparaffin are 1:(1 to 1.5). Further preferred weight ratios of C₁₂-C₁₅ alkyl benzoate to C₈-C₁₆ isoparaffin are (0.6 to 0.9):(2.5 to 3), in particular 0.8:3.

In further preferred oil mixtures containing 0 to a maximum of 2 wt. %, preferably 0 to a maximum of 1 wt. % of cyclomethicone, the two oil types ester/C₁₀-C₁₃ isoparaffin are included in equal percentages by weight. Further preferred weight ratios of ester to C₁₀₋₁₃ isoparaffin are (0.6 to 1):(1 to 3). Further preferred weight ratios of ester to C₁₀-C₁₃ isoparaffin are 1:(1 to 1.5). Further preferred weight ratios of ester to C₁₀-C₁₃ isoparaffin are (0.6 to 0.9):(2.5 to 3), in particular 0.8:3.

In further preferred oil mixtures containing 0 to a maximum of 2 wt. %, preferably 0 to a maximum of 1 wt. % of cyclomethicone, the two oil types isopropyl myristate/C₁₀-C₁₃ isoparaffin are included in equal percentages by weight. Further preferred weight ratios of isopropyl myristate to C₁₀₋₁₃ isoparaffin are (0.6 to 1):(1 to 3). Further preferred weight ratios of isopropyl myristate to C₁₀-C₁₃ isoparaffin are 1:(1 to 1.5). Further preferred weight ratios of isopropyl myristate to C₁₀-C₁₃ isoparaffin are (0.6 to 0.9):(2.5 to 3), in particular 0.8:3.

In further preferred oil mixtures containing 0 to a maximum of 2 wt. %, preferably 0 to a maximum of 1 wt. % of cyclomethicone, the two oil types isopropyl palmitate/C₁₀-C₁₃ isoparaffin are included in equal percentages by weight. Further preferred weight ratios of isopropyl palmitate to C₁₀₋₁₃ isoparaffin are (0.6 to 1):(1 to 3). Further preferred weight ratios of isopropyl palmitate to C₁₀-C₁₃ isoparaffin are 1:(1 to 1.5). Further preferred weight ratios of isopropyl palmitate to C₁₀-C₁₃ isoparaffin are (0.6 to 0.9):(2.5 to 3), in particular 0.8:3.

In further preferred oil mixtures containing 0 to a maximum of 2 wt. %, preferably 0 to a maximum of 1 wt. % of cyclomethicone, the two oil types 2-ethylhexyl palmitate/C₁₀-C₁₃ isoparaffin are included in equal percentages by weight. Further preferred weight ratios of 2-ethylhexyl palmitate to C₁₀₋₁₃ isoparaffin are (0.6 to 1):(1 to 3). Further preferred weight ratios of 2-ethylhexyl palmitate to C₁₀-C₁₃ isoparaffin are 1:(1 to 1.5). Further preferred weight ratios of 2-ethylhexyl palmitate to C₁₀-C₁₃ isoparaffin are (0.6 to 0.9):(2.5 to 3), in particular 0.8:3.

In further preferred oil mixtures containing 0 to a maximum of 2 wt. %, preferably 0 to a maximum of 1 wt. % of cyclomethicone, the two oil types C₁₂-C₁₅ alkyl benzoate/C₁₀-C₁₃ isoparaffin are included in equal percentages by weight. Further preferred weight ratios of C₁₂-C₁₅ alkyl benzoate to C₁₀₋₁₃ isoparaffin are (0.6 to 1):(1 to 3). Further preferred weight ratios of C₁₂-C₁₅ alkyl benzoate to C₁₀-C₁₃ isoparaffin are 1:(1 to 1.5). Further preferred weight ratios of C₁₂-C₁₅ alkyl benzoate to C₁₀-C₁₃ isoparaffin are (0.6 to 0.9):(2.5 to 3), in particular 0.8:3.

The compositions according to the invention are preferably present as a suspension, which means that the sweat-inhibiting active ingredient and optionally further insoluble constituents are suspended in a liquid carrier, which is optionally thickened or solidified into a stick.

Further particularly preferred deodorant or antiperspirant compositions according to the invention have the characterizing feature that the oil(s) that is/are liquid at 20° C. is/are included in a total amount from 0.1 to 80 wt. %, preferably 2 to 20 wt. %, particularly preferably 3 to 15 wt. %, relative in each case to the total weight of the composition.

Compositions that are particularly preferred according to the invention preferably contain furthermore at least one skin-cooling active ingredient. Skin-cooling active ingredients that are suitable according to the invention are for example menthol, isopulegol and menthol derivatives, for example menthyl lactate, menthyl glycolate, menthyl pyrrolidone carboxylic acid, menthyl methyl ether, menthoxypropanediol, menthone glycerol acetal (9-methyl-6-(1-methylethyl)-1,4-dioxaspiro(4.5)decane-2-methanol), monomenthyl succinate and 2-hydroxymethyl-3,5,5-trimethyl cyclohexanol. Menthol, isopulegol, menthyl lactate, menthoxypropanediol and menthyl pyrrolidone carboxylic acid as well as mixtures of these substances, in particular mixtures of menthol and menthyl lactate, menthol, menthol glycolate and menthyl lactate, menthol and menthoxypropanediol or menthol and isopulegol, are preferred as skin-cooling active ingredients.

It is particularly preferable according to the invention for at least one skin-cooling active ingredient to be included in a total amount from 0.01 to 1 wt. %, particularly preferably 0.02 to 0.5 wt. % and exceptionally preferably 0.05 to 0.2 wt. %, relative in each case to the total weight of the composition.

Compositions that are particularly preferred according to the invention that are packaged as a propellant-driven aerosol contain at least one propellant. Preferred propellants are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoro ethane, heptafluoro-n-propane, perfluoroethane, monochlorodifluoromethane, 1,1-difluoroethane, both individually and in combination. Hydrophilic propellants such as carbon dioxide for example can also advantageously be used within the meaning of the present invention if the chosen proportion of hydrophilic gases is small and lipophilic propellant (e.g. propane/butane) is present in excess. Propane, n-butane, isobutene and mixtures of these propellants are particularly preferred. It has been found that the use of n-butane as the sole propellant can be particularly preferred according to the invention.

The amount of propellant is preferably 20 to 80 wt. %, particularly preferably 30 to 70 wt. % and exceptionally preferably 40 to 50 wt. %, relative in each case to the total weight of the preparation, consisting of the composition according to the invention and the propellant.

Vessels made from metal (aluminum, white metal, tin), protected or shatterproof plastic or from glass externally coated with plastic are suitable as pressurized-gas containers, in the selection of which compressive strength and fracture strength, corrosion resistance, ease of filling and also aesthetic factors, manageability, printability, etc., play a part. Special internal protective coatings ensure corrosion resistance against the composition according to the invention.

Preferred compositions according to the invention contain furthermore at least one water-soluble polyhydric C₂-C₉ alkanol having 2 to 6 hydroxyl groups and/or at least one water-soluble polyethylene glycol having 3 to 20 ethylene oxides as well as mixtures thereof. These components are preferably selected from 1,2-propylene glycol, 2-methyl-1,3-propanediol, glycerol, butylene glycols such as 1,2-butylene glycol, 1,3-butylene glycol and 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol and mixtures of the aforementioned substances. Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20 and mixtures thereof, with PEG-3 to PEG-8 being preferred.

Preferred deodorant or antiperspirant compositions according to the invention have the characterizing feature that the at least one water-soluble polyhydric C₂-C₉ alkanol having 2 to 6 hydroxyl groups and/or at least one water-soluble polyethylene glycol having 3 to 20 ethylene oxide units is selected from 1,2-propylene glycol, 2-methyl-1,3-propanediol, glycerol, butylene glycols such as 1,2-butylene glycol, 1,3-butylene glycol and 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol and mixtures of the aforementioned substances.

Particularly preferred deodorant or antiperspirant compositions according to the invention have the characterizing feature that the at least one water-soluble polyhydric C₂-C₉ alkanol having 2 to 6 hydroxyl groups and/or at least one water-soluble polyethylene glycol having 3 to 20 ethylene oxide units is included in total in amounts from 3 to 30 wt. %, preferably 8 to 25 wt. %, particularly preferably 10 to 18 wt. %, relative in each case to the complete composition.

Further particularly preferred deodorant or antiperspirant compositions according to the invention have the characterizing feature that in order to improve the stick consistency and the sensory properties they additionally contain at least one solid, water-insoluble particulate filler other than talc and phyllosilicates. In an exceptionally preferred embodiment this filler is selected from optionally modified starches (obtained for example from corn, rice, potatoes) and starch derivatives, which are optionally pre-gelatinized, in particular starch derivatives of the DRY FLO® type, cellulose and cellulose derivatives, silicon dioxide, silicas, for example Aerosil® types, spherical polyalkyl sesquisiloxane particles (in particular Aerosil® R972 and Aerosil® 200V from Degussa), silica gels, boron nitride, lactoglobulin derivatives, for example sodium C₈₋₁₆ isoalkyl succinyl lactoglobulin sulfonate, obtainable from Brooks Industries as the commercial product Biopol® OE, glass powders, polymer powders, consisting in particular of polyolefins, polycarbonates, polyurethanes, polyamides, for example nylon, polyesters, polystyrenes, polyacrylates, (meth)acrylate or (meth)acrylate-vinylidene copolymers, which can be crosslinked, or silicones, as well as mixtures of these substances.

Polymer powders based on a polymethacrylate copolymer are available for example as the commercial product Polytrap® 6603 (Dow Corning). Other polymer powders, based for example on polyamides, are available under the name Orgasol® 1002 (polyamide 6) and Orgasol© 2002 (polyamide 12) from Elf Atochem. Further polymer powders that are suitable for the purpose according to the invention are for example polymethacrylates (Micropearl® M from SEPPIC or Plastic Powder A from NIKKOL), styrene-divinyl benzene copolymers (Plastic Powder FP from NIKKOL), polyethylene and polypropylene powders (ACCUREL® EP 400 from AKZO) or silicone polymers (Silicone Powder X2-1605 from Dow Corning).

Particularly preferred deodorant or antiperspirant compositions according to the invention have the characterizing feature that they contain at least one solid, water-insoluble particulate filler other than talc and phyllosilicates, in a total amount from 0.01 to 30 wt. %, preferably 5 to 20 wt. %, particularly preferably 8 to 15 wt. %, relative in each case to the complete composition.

Particularly preferred deodorant or antiperspirant compositions according to the invention have the characterizing feature that furthermore at least one fragrance component is included. Perfumes, perfume oils or perfume oil constituents can be used as the fragrance component. According to the invention perfume oils or fragrances can be individual aroma compounds, for example the synthetic products of the ester, ether, aldehyde, ketone, alcohol and hydrocarbon type. Aroma compounds of the ester type are for example benzyl acetate, phenoxyethyl isobutyrate, p-tert-butyl cyclohexyl acetate, linalyl acetate, dimethyl benzyl carbinyl acetate (DMBCA), phenylethyl acetate, benzyl acetate, ethyl methyl phenyl glycinate, allyl cyclohexyl propionate, styrallyl propionate, benzyl salicylate, cyclohexyl salicylate, floramat, melusat and j asmecyclate. The ethers include for example benzyl ethyl ethers and ambroxan, the aldehydes include for example linear alkanals having 8 to 18 C atoms, citral, citronellal, citronellyl oxyacetaldehyde, cyclamen aldehyde, lilial and bourgeonal, the ketones include for example ionones, alpha-isomethyl ionone and methyl cedryl ketone, the alcohols include anethol, citronellol, eugenol, geraniol, linalool, phenyl ethyl alcohol and terpineol, the hydrocarbons primarily include terpenes such as limonene and pinene. Mixtures of different aromas which together generate an attractive fragrance note are preferably used, however.

Particularly preferred deodorant or antiperspirant compositions according to the invention have the characterizing feature that at least one fragrance component is included in a total amount from 0.00001 to 4 wt. %, preferably 0.5 to 2 wt. %, relative in each case to the complete composition.

Advantageous constituents of the preparation according to the invention are disteardimonium hectorite, aluminum chlorohydrate, perfume and an aqueous suspension of at least one silver halide applied to a carrier, advantageously as an aerosol and advantageously with butane and/or propane as propellants.

The present application also provides a method for producing a sweat-inhibiting composition, wherein the phyllosilicate is mixed with the oil components and an aqueous suspension of at least one silver halide applied to a carrier, causing the gelling action of the phyllosilicate to be activated, perfume being added before or after gelation.

All that has been stated in respect of the compositions according to the invention applies with necessary alterations to further preferred embodiments of the production method according to the invention.

The present application also provides a non-therapeutic, cosmetic method for reducing and/or regulating sweat formation and/or body odor, in which the composition as described herein is applied in an effective amount to the skin, preferably to the skin in the armpit region.

All that has been stated in respect of the compositions according to the invention applies with necessary alterations to further preferred embodiments of the method according to the invention.

The present application also provides the non-therapeutic, cosmetic use of a sweat-inhibiting composition as described herein to reduce and/or regulate perspiration and/or body odor.

All that has been stated in respect of the compositions according to the invention applies with necessary alterations to further preferred embodiments of the uses according to the invention.

The examples below illustrate the preparations according to the invention. The proportions specified therein relate to the total mass of the preparation or active ingredient solution.

### EXAMPLES

With preferably butane/isobutane/propane as the propellant and a fill ratio of 5:95 to 30:70, preferably 10:90 to 20:80, preferably 15:85.

Antiperspirant Aerosols:

| | 1.1 | 1.2 | 1.3 |
|---|---|---|---|
| Isopropyl myristate | 2.34 | 3.20 | 3.10 |
| Persea gratissima oil | 0.10 | 0.15 | 0.10 |
| Disteardimonium hectorite | 4.00 | 3.80 | 3.90 |
| Aluminum chlorohydrate | 35.0 | 33.0 | 37.0 |
| Aqueous suspension of silver chloride | 0.7 | 0.3 | 0.1 |
| (2%) on TiO₂ (8%) with propylene glycol | | | |
| (5%), sodium di(2-ethylhexyl)sulfosuccinate | | | |
| (20%) and water (to 100%) | | | |
| Perfume | 5.33 | 5.0 | 5.1 |
| Sodium starch octenylsuccinate + mannitol | 2.67 | 0 | 1.23 |
| Cyclomethicone | to 100 | to 100 | to 100 |

TABLE 2
Suspensions for spraying as an antiperspirant spray

| | | 2.1 | 2.2 | 2.3 | 2.4 | 2.5 | 2.6 |
|---|---|---|---|---|---|---|---|
| Perfume | | 5.00 | 5.00 | 5.00 | 5.00 | 5.00 | 5.00 |
| Aluminum chlorohydrate | | 30.00 | 35.00 | 35.00 | 35.00 | 28.00 | 32.00 |
| (activated) | | | | | | | |
| Aqueous suspension of silver | | 0.6 | 0.6 | 0.3 | 0.2 | 0.4 | 0.4 |
| chloride (2%) on TiO₂ (8%) with | | | | | | | |
| propylene glycol (5%), sodium | | | | | | | |
| di(2-ethylhexyl)sulfosuccinate | | | | | | | |
| (20%) and water (to 100%) | | | | | | | |
| Encapsulated 2-benzyl heptan-1-ol | | 2.00 | 2.00 | 2.00 | — | — | — |
| (sodium starch octenylsuccinate + | | | | | | | |
| mannitol) | | | | | | | |
| Isopropyl palmitate | | 5.00 | 5.00 | — | — | — | 6.00 |
| C₁₂₋₁₅ alkyl benzoate | | — | — | 5.00 | 5.00 | — | — |
| Cosmacol PLG | | — | — | — | — | 5.00 | — |
| Disteardimonium hectorite | | 4.50 | 3.90 | 4.00 | 3.80 | 5.00 | 4.50 |
| Propylene carbonate | | 1.50 | 1.30 | 1.30 | 1.30 | 1.70 | 1.50 |
| Cyclopentasiloxane | | to 100 | to 100 | to 100 | to 100 | to 100 | to 100 |
| | 2.7 | 2.8 | 2.9 | 2.10 | 2.11 | 2.12 | 2.13 |
| Perfume | 5.00 | 5.00 | 5.00 | 5.00 | 5.00 | 5.00 | 5.00 |
| Aluminum | 32.00 | 28.00 | 30.00 | 35.00 | 32.00 | 32.00 | 28.00 |
| chlorohydrate | | | | | | | |
| (activated) | | | | | | | |
| Aqueous suspension of | 0.3 | 0.2 | 0.4 | 0.3 | 0.6 | 0.4 | 0.3 |
| silver chloride (2%) on | | | | | | | |
| TiO₂ (8%) with | | | | | | | |
| propylene glycol (5%), | | | | | | | |
| sodium di(2- | | | | | | | |
| ethylhexyl) | | | | | | | |
| sulfosuccinate (20%) | | | | | | | |
| and water (to 100%) | | | | | | | |
| Encapsulated 2-benzyl | 2.00 | — | — | — | — | 2.00 | — |
| heptan-1-ol (sodium | | | | | | | |
| starch octenylsuccinate + | | | | | | | |
| mannitol) | | | | | | | |
| Isopropyl myristate | | | | | | | 5.00 |
| Isopropyl palmitate | 5.00 | 5.00 | — | — | — | — | — |
| C₁₂₋₁₅ alkyl benzoate | — | — | 5.00 | 5.00 | — | — | — |
| Cosmacol PLG | — | — | — | — | 5.00 | 5.00 | — |
| Disteardimonium | 4.50 | 3.90 | 4.00 | 3.80 | 5.00 | 4.50 | 3.90 |
| hectorite | | | | | | | |
| Propylene carbonate | 1.50 | 1.30 | 1.30 | 1.30 | 1.70 | 1.50 | 1.30 |
| Tocopheryl acetate | 0.20 | — | 0.50 | 0.10 | — | — | — |
| Cyclopentasiloxane | to 100 | to 100 | to 100 | to 100 | to 100 | to 100 | to 100 |

Cosmacol PLG (INCI: DI-C12-13 ALKYL TARTRATE, TRI-C12-13 ALKYL CITRATE, SILICA)

| | 2.14 | 2.15 | 2.16 | 2.17 | 2.18 | 2.19 |
|---|---|---|---|---|---|---|
| Perfume | 5.00 | 5.00 | 5.00 | 5.00 | 5.00 | 5.00 |
| Aluminum chlorohydrate | 32.00 | 28.00 | 30.00 | 35.00 | 32.00 | 32.00 |
| (activated) | | | | | | |
| Aqueous suspension of silver | 0.6 | 0.3 | 0.6 | 0.4 | 0.3 | 0.3 |
| chloride (2%) on TiO₂ (8%) | | | | | | |
| with propylene glycol (5%), | | | | | | |
| sodium di(2- | | | | | | |
| ethylhexyl)sulfosuccinate | | | | | | |
| (20%) and water (to 100%) | | | | | | |
| Encapsulated 2-benzyl | 2.00 | 2.00 | — | 2.00 | 2.00 | — |
| heptan-1-ol (sodium starch | | | | | | |
| octenylsuccinate + mannitol) | | | | | | |
| Disteardimonium hectorite | 4.50 | 3.90 | 4.00 | 3.80 | 5.00 | 4.50 |
| Propylene carbonate | 1.50 | 1.30 | 1.30 | 1.30 | 1.70 | 1.50 |
| Tocopheryl acetate | 0.20 | — | 0.50 | 0.10 | — | — |
| C10-13 isoparaffin | 10.0 | 10.0 | 10.0 | 10.0 | 10.0 | 10.0 |
| Ethylhexyl palmitate | to 100 | to 100 | to 100 | — | — | — |
| Isopropyl myristate | — | — | — | to 100 | to 100 | to 100 |

The compositions 2.1 to 2.19 were introduced into spray cans made from aluminum coated on the inside and pressurized with an isobutane/butane/propane propellant mixture in the weight ratio of suspension to propellant of 25:75, 22:78, 20:80, 18:82, 15:85 and 13:87.

Antiperspirant Sticks:

| | 3.1 | 3.2 | 3.3 |
|---|---|---|---|
| Hydrogenated castor oil | 1.5 | 1.5 | 1.6 |
| Talc | 4.0 | 2.5 | 4.15 |
| PPG-14 Butyl ether | 15.0 | 14.43 | 0 |
| Caprylic/capric triglyceride | 0 | 0 | 14.5 |
| Aluminum chlorohydrate | 0 | 20.0 | 0 |
| Aluminum zirconium tetrachlorohydrex | 16.0 | 0 | 16.0 |
| GLY | | | |
| Stearyl alcohol | 20.0 | 21.0 | 20.5 |
| Aqueous suspension of silver chloride (2%) | 0.3 | 0.35 | 0.3 |
| on TiO₂ (8%) with propylene glycol (5%), | | | |
| sodium di(2-ethylhexyl)sulfosuccinate | | | |
| (20%) and water (to 100%) | | | |
| Perfume | 0.8 | 0.8 | 0.6 |
| Sodium starch octenylsuccinate + mannitol | 0.6 | 0.65 | 0.7 |
| Cyclomethicone | to 100 | to 100 | to 100 |

TABLE 4
Antiperspirant wax sticks

| | 4.1 | 4.2 | 4.3 | 4.4 | 4.5 |
|---|---|---|---|---|---|
| Hexyl decanol | 10.00 | 12.00 | 10.00 | 8.00 | 8.00 |
| PPG-14 Butyl ether | 6.00 | 5.00 | 6.00 | 8.00 | 8.00 |
| Hydrogenated castor oil | 4.00 | 5.00 | 6.00 | 5.00 | 5.00 |
| Stearyl alcohol | 12.00 | 14.00 | 11.00 | 16.00 | 16.00 |
| Cetyl alcohol | 6.00 | 5.00 | 6.00 | 3.00 | 3.00 |
| PEG-20 Glyceryl stearate | 5.00 | 4.00 | 6.00 | 4.00 | 4.00 |
| Ceteareth-30 | 3.00 | 1.00 | 3.00 | — | — |
| Perfume | 1.00 | 1.20 | 0.80 | 1.00 | 1.00 |
| Aluminum chlorohydrate | 20.00 | 20.00 | 18.00 | — | — |
| (Reach 103) | | | | | |
| Aluminum zirconium | — | — | — | 23.00 | 23.00 |
| tetrachlorohydrate Gly | | | | | |
| (Reach AZP 908 | | | | | |
| Powder Superultrafine) | | | | | |
| Aqueous suspension | 0.7 | 0.3 | 0.6 | 0.4 | 0.3 |
| of silver chloride (2%) | | | | | |
| on TiO₂ (8%) with | | | | | |
| propylene glycol (5%), | | | | | |
| sodium di(2- | | | | | |
| ethylhexyl)sulfosuccinate | | | | | |
| (20%) and water | | | | | |
| (to 100%) | | | | | |
| Talc | 8.00 | 5.00 | 8.00 | 7.00 | 7.00 |
| Cyclopentasiloxane | to 100 | to 100 | to 100 | to 100 | to 100 |

TABLE 5
Anhydrous antiperspirant wax sticks without cyclomethicone

| | 5.1 | 5.2 | 5.3 | 5.4 | 5.5 | 5.6 |
|---|---|---|---|---|---|---|
| Stearyl alcohol | 16 | 18 | 18 | 18 | 18 | 18 |
| Behenyl alcohol | — | — | 0.2 | — | — | — |
| Ceteareth-30 | 3 | 3 | 3 | 3 | 3 | 3 |
| PPG-14 Butyl ether | 15 | 5 | 10 | 15 | 10 | 7.5 |
| Hydrogenated polydecene | — | — | — | — | 15 | 15 |
| Cocoglycerides (FP 30-32° C.) | 5 | 4 | 4 | 4 | 4 | 4 |
| Hydrogenated castor oil | 1.5 | 1.5 | 2 | 1.5 | 1.5 | 1.5 |
| Aluminum chlorohydrate | 20 | 20 | 20 | 20 | 20 | 20 |
| Aqueous suspension of silver | 0.7 | 0.3 | 0.6 | 0.4 | 0.3 | 0.5 |
| chloride (2%) on TiO₂ (8%) | | | | | | |
| with propylene glycol (5%), | | | | | | |
| sodium di(2- | | | | | | |
| ethylhexyl)sulfosuccinate | | | | | | |
| (20%) and water (to 100%) | | | | | | |
| Talc | 5 | 3 | 5 | 3 | 3 | 5 |
| Perfume | 1 | 1 | 1 | 1 | 1 | 1 |
| Dimethicone (2 cSt) | — | — | 18.4 | 23 | to 100 | to 100 |
| Dimethicone (5 cSt) | to 100 | to 100 | to 100 | to 100 | — | — |

TABLE 6
Antiperspirant cream anhydrous (soft solid)

| | 6.1 | 6.2 | 6.3 | 6.4 |
|---|---|---|---|---|
| Aluminum chlorohydrate | 20.00 | 22.00 | 20.00 | |
| Aluminum zirconium | — | — | — | 24.00 |
| tetrachlorohydrate Gly | | | | |
| Hexyl decanol | 5.00 | 4.50 | 5.50 | 6.00 |
| Dicaprylyl ether | 3.00 | 4.00 | — | — |
| Cocoglycerides | 5.00 | 6.00 | 7.00 | 3.00 |
| C18-C36 Triglyceride | 6.00 | 5.00 | 4.00 | 3.00 |
| Ceteareth-30 | 3.00 | 2.00 | 2.50 | 4.00 |
| PEG-20 Glyceryl stearate | 5.00 | 6.00 | 3.00 | 2.00 |
| Cellulose | 3.00 | 2.00 | 5.00 | 1.00 |
| Aluminum starch octenylsuccinate | 5.00 | 4.00 | 6.00 | 5.00 |
| Silica | 1.00 | 2.00 | 0.50 | — |
| Aqueous suspension of silver | 0.5 | 0.3 | 0.4 | 0.7 |
| chloride (2%) on TiO₂ (8%) with | | | | |
| propylene glycol (5%), sodium | | | | |
| di(2-ethylhexyl)sulfosuccinate | | | | |
| (20%) and water (to 100%) | | | | |
| Talc | 10.00 | 5.00 | 7.00 | 12.00 |
| Allantoin | 0.10 | 0.10 | — | — |
| Perfume | 1.00 | 1.50 | 2.00 | 0.80 |
| Cyclopentasiloxane | to 100 | to 100 | — | — |
| C10-13 isoparaffin | — | — | to 100 | to 100 |

TABLE 7
Anhydrous antiperspirant roll-on compositions

| | 7.1 | 7.2 | 7.3 | 7.4 | 7.5 |
|---|---|---|---|---|---|
| Quaternium-18 hectorite | 4.0 | 2.5 | 2.5 | 3.2 | 3.6 |
| Perfume | 0.5 | 0.5 | 0.5 | 1.0 | 1.0 |
| Perfume (encapsulated) | 0.5 | 0.5 | 0.5 | 1.0 | 1.0 |
| Al/Zr tetrachlorohydrate | 20 | 20 | 20 | — | — |
| gly (activated) | | | | | |
| Al/Zr pentachlorohydrate | — | — | — | 20 | 20 |
| gly (activated) | | | | | |
| Aqueous suspension | 0.4 | 0.2 | 0.2 | 0.3 | 0.3 |
| of silver chloride (2%) | | | | | |
| on TiO₂ (8%) | | | | | |
| with propylene glycol | | | | | |
| (5%), sodium di(2- | | | | | |
| ethylhexyl)sulfosuccinate | | | | | |
| (20%) and water | | | | | |
| (to 100%) | | | | | |
| Propylene carbonate | 1.0 | 1.0 | — | — | 1.0 |
| Dimethicone (50 cSt) | — | — | 5.0 | 3.0 | |
| Aerosil 300 | — | 0.2 | 0.4 | — | — |
| (fumed silica) | | | | | |
| Cyclomethicone | to 100 | to 100 | to 100 | to 100 | to 100 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An anhydrous cosmetic or dermatological preparation, comprising:
an aqueous suspension of at least one silver halide having a water content of up to 90 wt. %, relative to the total mass of the suspension, applied to a carrier, in combination with
one or more passivating agents selected from the group consisting of phyllosilicates and talc,
one or more antiperspirant active ingredients, and perfume.

2. The preparation according to claim 1, wherein the aqueous suspension of at least one silver halide comprises silver chloride.

3. The preparation according to claim 1, wherein the carrier comprises titanium dioxide.

4. The composition according to claim 1, wherein the aqueous suspension further comprises, relative to the weight of the suspension, 0.1 to 30 wt. % of at least one sulfosuccinate.

5. The composition according to claim 3, wherein the aqueous suspension comprises, relative to the weight of the suspension, 0.1 to 30 wt. % of sodium di(2-ethylhexyl)sulfosuccinate

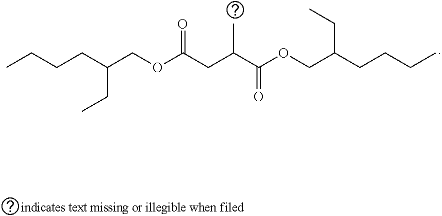

6. The composition according to claim 1, wherein the aqueous suspension comprises, relative to the weight of the suspension, 0.1 to 10 wt. % of propylene glycol.

7. The composition according to claim 3, wherein the aqueous suspension comprises, relative to the weight of the suspension, 0.1 to 20 wt. % of titanium dioxide.

8. The composition according to claim 1, wherein the composition is propellant-free and wherein silver ions (Ag⁺) are included in a total amount from 1 to 100 ppm relative to the weight of the composition.

9. The composition according to claim 2, wherein the composition is propellant-free and wherein silver chloride is included in a total amount from 1.3 to 133 ppm relative to the weight of the composition.

10. The composition according to claim 1, wherein the composition is propellant-free and wherein the aqueous suspension of at least one silver halide comprises silver bromide in a total amount from 1.7 to 174 ppm relative to the weight of the composition.

11. The preparation according to claim 1, wherein the passivating agent for the phyllosilicates comprises at least one clay mineral selected from the group consisting of bentonite, montmorillonite, nontronite, hectorite, saponite, sauconite, beidellite, allevardite, illite, halloysite, attapulgite, muscovite, serpentine, kaolinite, pyrophyllite, smectite, glauconite, vermiculite and sepiolite.

12. The preparation according to claim 11, wherein the passivating agent for the phyllosilicates comprises disteardimonium hectorite.

13. A method for producing a sweat-inhibiting composition comprising:
mixing a phyllosilicate with any oil components and an aqueous suspension, causing gellation of the phyllosilicate to be activated,
wherein the aqueous suspension is a suspension of at least one silver halide having a water content of up to 90 wt. %, relative to the total mass of the suspension, applied to a carrier, in combination with
one or more passivating agents selected from the group consisting of phyllosilicates and talc,
one or more antiperspirant active ingredients; and
adding a perfume to the mixed components before or after gelation.

14. A non-therapeutic, cosmetic method for reducing and/or regulating sweat formation and/or body odor, comprising:
applying a composition according to claim 1 in an effective amount to the skin.
